(19) United States
(12) Patent Application Publication
(10) Pub. No.: US 2025/0170108 A1
(43) Pub. Date: May 29, 2025

(54) CHEMOTHERAPY FOR CANCER USING AZABICYCLO COMPOUND

(71) Applicant: TAIHO PHARMACEUTICAL CO., LTD., Tokyo (JP)

(72) Inventor: Shuichi OHKUBO, Chiyoda-ku (JP)

(73) Assignee: TAIHO PHARMACEUTICAL CO., LTD., Tokyo (JP)

(21) Appl. No.: 19/031,720

(22) Filed: Jan. 18, 2025

Related U.S. Application Data
(63) Continuation of application No. 16/627,146, filed on Dec. 27, 2019.

(30) Foreign Application Priority Data
Jun. 30, 2017 (JP) 2017-128823
Sep. 15, 2017 (JP) 2017-177390
May 24, 2018 (JP) 2018-099931

(51) Int. Cl. A61K 31/437; A61K 39/395; A61P 35/00; A61P 37/04

(52) U.S. Cl. CPC A61K 31/437; A61K 39/39558; A61K 39/39566; A61P 35/00; A61P 37/04

(57) ABSTRACT

Provided are a novel cancer treatment method and immunostimulant which exhibit a remarkably excellent antitumor effect with little side effects. An antitumor agent and an immunostimulant include an azabicyclo compound and an immune checkpoint molecule regulator which are administered in combination.

This application is a continuation of U.S. application Ser. No. 16/627,146, filed on Dec. 27, 2019, pending, which is a 371 application of PCT/JP2018/024776 filed Jun. 29, 2018, and claims the benefit of Japanese Application No. 2017-128823, filed Jun. 30, 2017, Japanese Application No. 2017-177390, filed Sep. 15, 2017, and Japanese Application No. 2018-099931, filed May 24, 2018. The contents of each of these applications are incorporated herein by reference in their entirety.

### TECHNICAL FIELD

The present invention relates to an antitumor agent comprising an azabicyclo compound or a salt thereof in combination with an immune checkpoint molecule regulator; an antitumor effect enhancing agent and a kit preparation; and an immunostimulant containing an azabicyclo compound or a salt thereof as an active ingredient.

### BACKGROUND ART

A group of proteins called molecular chaperons is multifunctional in such a way that they promote the formation of the functional structures of other proteins or maintain these structures, promote correct association, inhibit unnecessary aggregation, protect other proteins from degradation, and promote secretion (Non Patent Literature 1). HSP90 is a molecular chaperon as abundant as approximately 1 to 2% of all intracellular soluble proteins and is however unnecessary for the biosynthesis of the majority of polypeptides, unlike other chaperon proteins (Non Patent Literature 1). Signaling-related factors (e.g., ERBB1/EGFR, ERBB2/HER2, MET, IGF1R, KDR/VEGFR, FLT3, ZAP70, KIT, CHUK/IKK, BRAF, RAF1, SRC and AKT), cell cycle regulators (e.g., CDK4, CDK6, Cyclin D, PLK1 and BIRC5), and transcriptional regulators (e.g., HIF-1α, p53, androgen receptor, estrogen receptor and progesterone receptor) are known as the main client proteins whose structure formation or stability is regulated by HSP90 through the interaction therebetween (Non Patent Literatures 2 and 3). HSP90 is deeply involved in cell proliferation or survival by maintaining the normal functions of these proteins. Furthermore, HSP90 is required for the normal functions of mutated or chimeric factors (e.g., BCR-ABL and NPM-ALK) which cause carcinogenesis or exacerbation of cancer. This indicates the importance of HSP90 particularly for processes such as carcinogenesis, cancer survival, growth, exacerbation and metastasis (Non Patent Literature 2).

The inhibition of the chaperon functions of HSP90 by specific inhibitors such as geldanamycin causes the inactivation, destabilization and degradation of the client proteins, resulting in induction of a halt in cell proliferation or apoptosis (Non Patent Literature 4). In terms of the physiological functions of HSP90, HSP90 inhibitors are characterized in that they can simultaneously inhibit a plurality of signaling pathways involved in cancer survival/growth. Thus, the HSP90 inhibitors can serve as drugs having extensive and effective anticancer activity. Moreover, from the findings that cancer cell-derived HSP90 has higher activity and higher affinity for ATP or inhibitors than those of normal cell-derived HSP90, it has been expected that the HSP90 inhibitors would serve as drugs having high cancer selectivity (Non Patent Literature 5).

On the other hand, the immunological system is an important mechanism for self-defense against various diseases caused by in vivo and in vitro factors. Deterioration of the functions of the immunological system has pathologically adverse effects such as development of infectious diseases by bacteria and viruses, development of tumors, and delay of recovery from injuries and diseases. Therefore, activation of the immunological system is very important for prevention and treatment of various diseases. As one of new methods for treating cancer, cancer immunotherapy is being developed.

Activation of adaptive immunological reaction is initiated by binding of an antigenic peptide-MHC complex to a T-cell receptor (TCR). Further, this binding is regulated by costimulation or coinhibition due to binding between the B7 family which is a costimulatory molecule and the CD28 family which is a receptor of the B7 family. That is, for T-cells to be activated in an antigen-specific manner, two characteristic signal transduction events are required, and T-cells which have not undergone costimulation from the B7 family and have undergone only antigen-stimulation are turned into a nonresponsive state (anergy), so that immunological tolerance is induced in the T-cells.

By taking advantage of this mechanism, cancer cells suppress activation of antigen-specific T-cells to escape from the immune surveillance, and continuously grow. Thus, for cancer treatment, it is considered effective that by enhancement of costimulation and blocking of coinhibition, an in vivo antitumor immune response is induced in a cancer patient to control immune escape of a tumor, and various cancer immunotherapies targeted at costimulatory molecules (stimulatory costimulatory molecules) or coinhibitory molecules (inhibitory costimulatory molecules) have been proposed (Patent Literature 6). For example, as an immune checkpoint molecule regulator for activating T-cells by inhibiting binding of PD-1 and ligands thereof (PD-L1 and PD-L2), nivolumab (human-type IgG4 monoclonal antibody against human PD-1) is used for malignant melanoma etc. (Patent Literature 1 and Non Patent Literature 7).

An azabicyclo compound represented by the general formula (I) below or a salt thereof is known as a HSP90 inhibitor (Patent Literature 2), and a combination of the HSP90 inhibitor and another antitumor agent has been heretofore reported (Patent Literature 3).

However, the HSP90 inhibitor and an immune checkpoint molecule regulator have not been used in combination yet. In addition, the immunostimulatory action of the HSP90 inhibitor is not known.

### CITATION LIST

### Patent Literature

Patent Literature 1: International Publication No. WO2004/004771
Patent Literature 2: International Publication No. WO2011/004610
Patent Literature 3: International Publication No. WO2015/046498

### Non Patent Literature

Non Patent Literature 1: Nat. Rev. Cancer, 5: 761-772 (2005)
Non Patent Literature 2: TRENDS Mol. Med., 10: 283-290 (2004)
Non Patent Literature 3: Clin. Cancer Res., 15: 9-14 (2009)
Non Patent Literature 4: Curr. Opin. Pharmacol., 8: 370-374 (2008)
Non Patent Literature 5: Drug Resist. Updat., 12: 17-27 (2009)
Non Patent Literature 6: Nat. Rev. Cancer, 12: 252-264 (2012)
Non patent Literature 7: N. Engl. J. Med., 366: 2443-2454 (2012)

### SUMMARY OF THE INVENTION

### Technical Problem

An object of the present invention is to provide a novel cancer treatment method which exhibits a remarkably excellent antitumor effect with little side effects.

### Solution to Problem

The present inventor has combined an azabicyclo compound represented by the general formula (I) and an immune checkpoint molecule regulator, and has studied the antitumor effect of the combination. As a result, the present inventor has found that the antitumor effect is more remarkably enhanced without causing severe side effects as compared to a case where a single agent is used.

In addition, the present inventor has found that the azabicyclo compound has immunostimulatory action.

Specifically, the present invention provides the following aspects [1] to [64].
[1] An antitumor agent comprising azabicyclo compound or a salt thereof and an immune checkpoint molecule regulator which are administered in combination, the azabicyclo compound being represented by the following general formula (I):

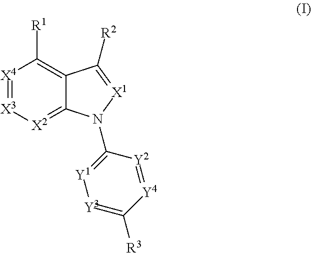

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[2] The antitumor agent according to [1], wherein the azabicyclo compound is a compound of the general formula (I) in which X¹ is CH or N;
X² is N, and X³ and X⁴ are CH;
Y¹ and Y³ are CH, one or two of Y² and Y⁴ are C—R⁴, and the other is CH;
R¹ is any one of an optionally substituted 1H-imidazol-1-yl group, an optionally substituted pyrazol-4-yl group, an optionally substituted thiophen-3-yl group, an optionally substituted furan-2-yl group, an optionally substituted pyridin-3-yl group, an optionally substituted pyridin-4-yl group, an optionally substituted indol-5-yl group, an optionally substituted 1H-pyrrolo[2,3-b]pyridin-5-yl group, an optionally substituted benzofuran-2-yl group, an optionally substituted quinolin-3-yl group and an optionally substituted 5,6,7,8-tetrahydroquinon-3-yl group;
R² is an alkyl group having 1 to 6 carbon atoms and optionally having a halogen atom, or an alkenyl group having 2 to 6 carbon atoms;
R³ is —CO—R⁵;
R⁴ is an alkyl group having 1 to 6 carbon atoms and optionally having a halogen atom, a mono- or di-(C1-C6 alkyl)amino group or a monocyclic 5- to 7-membered saturated heterocyclic group having 1 or 2 heteroatoms selected from the group consisting of N, S and O, an alkoxy group having 1 to 6 carbon atoms, —N(R⁶)(R⁷), —S—R³ or —CO—R⁹;
R⁵ is an amino group, or a mono- or di-(C1-C6 alkyl)amino group;
R⁶ is a hydrogen atom, or an optionally substituted alkyl group having 1 to 6 carbon atoms;
R⁷ is a hydrogen atom, an optionally substituted alkyl group having 1 to 6 carbon atoms, an optionally substituted cycloalkyl group having 3 to 7 carbon atoms, an optionally substituted aralkyl group having 7 to 12 carbon atoms, an optionally substituted aromatic hydrocarbon group having 6 to 14 carbon atoms, an optionally substituted monocyclic or dicyclic saturated heterocyclic group having 1 to 4 heteroatoms selected from the group consisting of N, S and O, or an optionally substituted monocyclic or dicyclic unsaturated heterocyclic group having 1 to 4 heteroatoms selected from the group consisting of N, S and O, or R⁶ and R⁷ form a 5- to 7-membered saturated heterocyclic group, together with the nitrogen atom to which they are attached;
R⁸ is an optionally substituted cycloalkyl group having 3 to 7 carbon atoms, or an optionally substituted aromatic hydrocarbon group having 6 to 14 carbon atoms; and
R⁹ is a hydrogen atom, a hydroxyl group, an amino group, or a mono- or a di-(C1-C6 alkyl)amino group.
[3] The antitumor agent according to [1] or [2], wherein the azabicyclo compound is 3-ethyl-4-{3-isopropyl-4-(4-(1-methyl-1H-pyrazol-4-yl)-1H-imidazol-1-yl)-1H-pyrazolo[3,4-b]pyridin-1-yl}benzamide.
[4] The antitumor agent according to any one of [1] to [3], wherein the immune checkpoint molecule regulator is at least one or more selected from the group consisting of a PD-1 pathway antagonist, an ICOS pathway agonist, a CTLA-4 pathway antagonist and a CD28 pathway agonist.
[5] The antitumor agent according to any one of [1] to [4], wherein the immune checkpoint molecule regulator is at least one or more selected from the group consisting of a PD-1 pathway antagonist, a CTLA-4 pathway antagonist and a CD28 pathway agonist.
[6] The antitumor agent according to any one of [1] to [5], wherein the immune checkpoint molecule regulator is at least one or more selected from the group consisting of a PD-1 pathway antagonist and a CTLA-4 pathway antagonist.
[7] The antitumor agent according to any one of [1] to [6], wherein the immune checkpoint molecule regulator is a PD-1 pathway antagonist.
[8] The antitumor agent according to any one of [4] to [7], wherein the PD-1 pathway antagonist is at least one or more selected from the group consisting of an anti-PD-1 antibody, an anti-PD-L1 antibody and an anti-PD-L2 antibody.
[9] The antitumor agent according to [8], wherein the anti PD-1-antibody is at least one or more selected from the group consisting of nivolumab and pembrolizumab, and the anti-PD-L1 antibody is at least one or more selected from the group consisting of atezolizumab, durvalumab and avelumab.
[10] The antitumor agent according to any one of [4] to [6], wherein the CTLA-4 pathway antagonist is an anti-CTLA-4 antibody.
[11] The antitumor agent according to [10], wherein the anti-CTLA-4 antibody is at least one or more selected from the group consisting of ipilimumab and tremelimumab.
[12] An antitumor effect enhancing agent for an immune checkpoint molecule regulator, comprising an azabicyclo compound or a salt thereof as an active ingredient, the azabicyclo compound being represented by the following general formula (I):

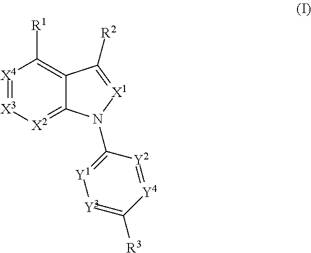

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R⁸ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R⁸ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[13] An antitumor agent for treating a cancer patient given an immune checkpoint molecule regulator, the antitumor agent comprising an azabicyclo compound or a salt thereof,
the azabicyclo compound being a compound represented by the general formula (I):

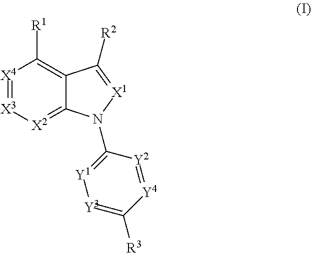

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[14] An antitumor agent for treating a cancer patient given an azabicyclo compound or a salt thereof, the antitumor agent comprising an immune checkpoint molecule regulator,
the azabicyclo compound being a compound represented by the general formula (I):

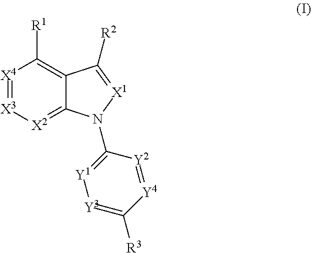

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[15] An antitumor agent comprising an azabicyclo compound or a salt in combination with an immune checkpoint molecule regulator,
the azabicyclo compound being a compound represented by the general formula (I):

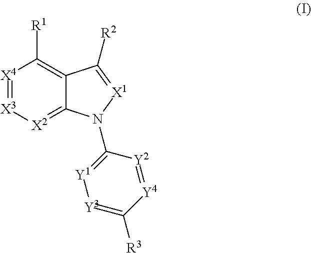

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[16] An immunostimulant comprising an azabicyclo compound or a salt thereof as an active ingredient, the azabicyclo compound being represented by the general formula (I):

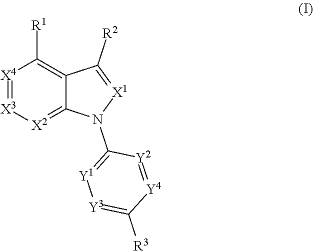

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[17] An azabicyclo compound or a salt thereof for use in treating a tumor by administration thereof in combination with an immune checkpoint molecule regulator, the azabicyclo compound being represented by the general formula (I):

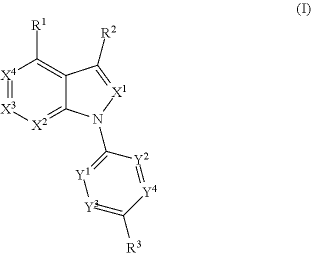

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[18] The azabicyclo compound according to [17] or a salt thereof, wherein the azabicyclo compound is a compound of the general formula (I) in which X¹ is CH or N;
X² is N, and X³ and X⁴ are CH;
Y¹ and Y³ are CH, one or two of Y² and Y⁴ are C—R⁴, and the other is CH;
R¹ is any one of an optionally substituted 1H-imidazol-1-yl group, an optionally substituted pyrazol-4-yl group, an optionally substituted thiophen-3-yl group, an optionally substituted furan-2-yl group, an optionally substituted pyridin-3-yl group, an optionally substituted pyridin-4-yl group, an optionally substituted indol-5-yl group, an optionally substituted 1H-pyrrolo[2,3-b]pyridin-5-yl group, an optionally substituted benzofuran-2-yl group, an optionally substituted quinolin-3-yl group and an optionally substituted 5,6,7,8-tetrahydroquinon-3-yl group;
R² is an alkyl group having 1 to 6 carbon atoms and optionally having a halogen atom, or an alkenyl group having 2 to 6 carbon atoms;
R³ is —CO—R⁵;
R⁴ is an alkyl group having 1 to 6 carbon atoms and optionally having a halogen atom, a mono- or di-(C1-C6 alkyl)amino group or a monocyclic 5- to 7-membered saturated heterocyclic group having 1 or 2 heteroatoms selected from the group consisting of N, S and O, an alkoxy group having 1 to 6 carbon atoms, —N(R⁶)(R⁷), —S—R³ or —CO—R⁹;
R⁵ is an amino group, or a mono- or di-(C1-C6 alkyl)amino group;
R⁶ is a hydrogen atom, or an optionally substituted alkyl group having 1 to 6 carbon atoms;
R⁷ is a hydrogen atom, an optionally substituted alkyl group having 1 to 6 carbon atoms, an optionally substituted cycloalkyl group having 3 to 7 carbon atoms, an optionally substituted aralkyl group having 7 to 12 carbon atoms, an optionally substituted aromatic hydrocarbon group having 6 to 14 carbon atoms, an optionally substituted monocyclic or dicyclic saturated heterocyclic group having 1 to 4 heteroatoms selected from the group consisting of N, S and O, or an optionally substituted monocyclic or dicyclic unsaturated heterocyclic group having 1 to 4 heteroatoms selected from the group consisting of N, S and O, or R⁶ and R⁷ form a 5- to 7-membered saturated heterocyclic group, together with the nitrogen atom to which they are attached;
R⁸ is an optionally substituted cycloalkyl group having 3 to 7 carbon atoms, or an optionally substituted aromatic hydrocarbon group having 6 to 14 carbon atoms; and
R⁹ is a hydrogen atom, a hydroxyl group, an amino group, or a mono- or a di-(C1-C6 alkyl) amino group.
[19] The azabicyclo compound according to [17] or [18] or a salt thereof, wherein the azabicyclo compound is 3-ethyl-4-{3-isopropyl-4-(4-(1-methyl-1H-pyrazol-4-yl)-1H-imidazol-1-yl)-1H-pyrazolo[3,4-b]pyridin-1-yl}benzamide.
[20] The azabicyclo compound according to any one of [17] to or a salt thereof, wherein the immune checkpoint molecule regulator is at least one or more selected from the group consisting of a PD-1 pathway antagonist, an ICOS pathway agonist, a CTLA-4 pathway antagonist and a CD28 pathway agonist.
[21] The azabicyclo compound according to any one of [17] to or a salt thereof, wherein the immune checkpoint molecule regulator is at least one or more selected from the group consisting of a PD-1 pathway antagonist, a CTLA-4 pathway antagonist and a CD28 pathway agonist.
[22] The azabicyclo compound according to any one of [17] to or a salt thereof, wherein the immune checkpoint molecule regulator is at least one or more selected from the group consisting of a PD-1 pathway antagonist and a CTLA-4 pathway antagonist.
[23] The azabicyclo compound according to any one of [17] to or a salt thereof, wherein the immune checkpoint molecule regulator is a PD-1 pathway antagonist.
[24] The azabicyclo compound according to any one of [20] to or a salt thereof, wherein the PD-1 pathway antagonist is at least one or more selected from the group consisting of an anti-PD-1 antibody, an anti-PD-L1 antibody and an anti-PD-L2 antibody.
[25] The azabicyclo compound according to [24] or a salt thereof, wherein the anti PD-1-antibody is at least one or more selected from the group consisting of nivolumab and pembrolizumab, and the anti-PD-L1 antibody is at least one or more selected from the group consisting of atezolizumab, durvalumab and avelumab.
[26] The azabicyclo compound according to any one of [20] to or a salt thereof, wherein the CTLA-4 pathway antagonist is an anti-CTLA-4 antibody.
[27] The azabicyclo compound according to [26] or a salt thereof, wherein the anti-CTLA-4 antibody is at least one or more selected from the group consisting of ipilimumab and tremelimumab.
[28] An azabicyclo compound or a salt thereof for use in enhancing the antitumor effect of an immune checkpoint molecule regulator, the azabicyclo compound being represented by the general formula (I):

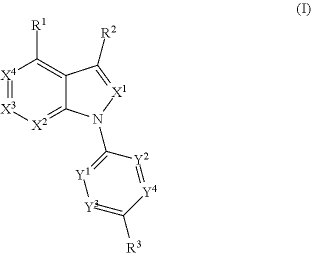

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[29] An azabicyclo compound or a salt thereof for use in treating a tumor in a cancer patient given an immune checkpoint molecule regulator, the azabicyclo compound being represented by the general formula (I):

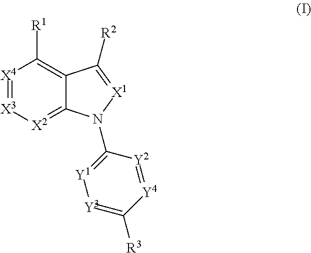

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R⁸ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R⁸ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[30] An immune checkpoint molecule regulator for use in treating a tumor in a cancer patient given an azabicyclo compound or a salt thereof, the azabicyclo compound being a compound represented by the general formula (I):

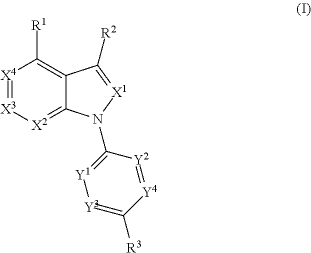

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[31] A combination of an azabicyclo compound or a salt thereof and an immune checkpoint molecule regulator for use in treating a tumor, the azabicyclo compound being represented by the general formula (I):

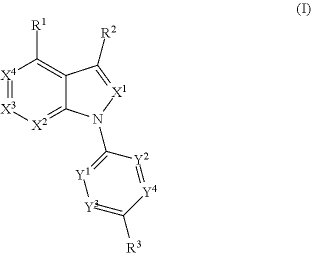

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[32] An azabicyclo compound or a salt thereof for use in immunostimulation, the azabicyclo compound being represented by the general formula (I):

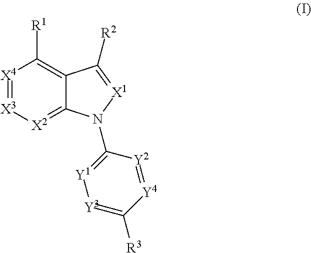

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[33] Use of an azabicyclo compound or a salt thereof for producing an antitumor agent which is administered in combination with an immune checkpoint molecule regulator, the azabicyclo compound being represented by the general formula (I):

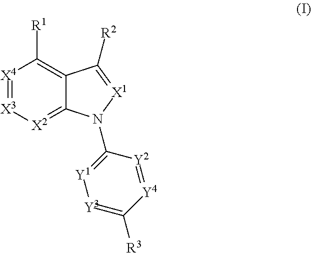

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[34] The use of an azabicyclo compound according to [33] or a salt thereof, wherein the azabicyclo compound is a compound of the general formula (I) in which X¹ is CH or N;
X² is N, and X³ and X⁴ are CH;
Y¹ and Y³ are CH, one or two of Y² and Y⁴ are C—R⁴, and the other is CH;
R¹ is any one of an optionally substituted 1H-imidazol-1-yl group, an optionally substituted pyrazol-4-yl group, an optionally substituted thiophen-3-yl group, an optionally substituted furan-2-yl group, an optionally substituted pyridin-3-yl group, an optionally substituted pyridin-4-yl group, an optionally substituted indol-5-yl group, an optionally substituted 1H-pyrrolo[2,3-b]pyridin-5-yl group, an optionally substituted benzofuran-2-yl group, an optionally substituted quinolin-3-yl group and an optionally substituted 5,6,7,8-tetrahydroquinon-3-yl group;
R² is an alkyl group having 1 to 6 carbon atoms and optionally having a halogen atom, or an alkenyl group having 2 to 6 carbon atoms;
R³ is —CO—R⁵;
R⁴ is an alkyl group having 1 to 6 carbon atoms and optionally having a halogen atom, a mono- or di-(C1-C6 alkyl)amino group or a monocyclic 5- to 7-membered saturated heterocyclic group having 1 or 2 heteroatoms selected from the group consisting of N, S and O, an alkoxy group having 1 to 6 carbon atoms, —N(R⁶)(R⁷), —S—R³ or —CO—R⁹;
R⁵ is an amino group, or a mono- or di-(C1-C6 alkyl)amino group;
R⁶ is a hydrogen atom, or an optionally substituted alkyl group having 1 to 6 carbon atoms;
R⁷ is a hydrogen atom, an optionally substituted alkyl group having 1 to 6 carbon atoms, an optionally substituted cycloalkyl group having 3 to 7 carbon atoms, an optionally substituted aralkyl group having 7 to 12 carbon atoms, an optionally substituted aromatic hydrocarbon group having 6 to 14 carbon atoms, an optionally substituted monocyclic or dicyclic saturated heterocyclic group having 1 to 4 heteroatoms selected from the group consisting of N, S and O, or an optionally substituted monocyclic or dicyclic unsaturated heterocyclic group having 1 to 4 heteroatoms selected from the group consisting of N, S and O, or R⁶ and R⁷ form a 5- to 7-membered saturated heterocyclic group, together with the nitrogen atom to which they are attached;
R⁸ is an optionally substituted cycloalkyl group having 3 to 7 carbon atoms, or an optionally substituted aromatic hydrocarbon group having 6 to 14 carbon atoms; and
R⁹ is a hydrogen atom, a hydroxyl group, an amino group, or a mono- or a di-(C1-C6 alkyl)amino group.
[35] The use of an azabicyclo compound or a salt thereof according to [33] or [34], wherein the azabicyclo compound is 3-ethyl-4-{3-isopropyl-4-(4-(1-methyl-1H-pyrazol-4-yl)-1H-imidazol-1-yl)-1H-pyrazolo[3,4-b]pyridin-1-yl}benzamide.
[36] The use of an azabicyclo compound or a salt thereof as according to any one of [33] to [35], wherein the immune checkpoint molecule regulator is at least one or more selected from the group consisting of a PD-1 pathway antagonist, an ICOS pathway agonist, a CTLA-4 pathway antagonist and a CD28 pathway agonist.
[37] The use of an azabicyclo compound or a salt thereof as according to any one of [33] to [36], wherein the immune checkpoint molecule regulator is at least one or more selected from the group consisting of a PD-1 pathway antagonist, a CTLA-4 pathway antagonist and a CD28 pathway agonist.
[38] The use of an azabicyclo compound or a salt thereof as according to any one of [33] to [37], wherein the immune checkpoint molecule regulator is at least one or more selected from the group consisting of a PD-1 pathway antagonist and a CTLA-4 pathway antagonist.
[39] The use of an azabicyclo compound or a salt thereof according to any one of [33] to [38], wherein the immune checkpoint molecule regulator is a PD-1 pathway antagonist.
[40] The use of an azabicyclo compound or a salt thereof according to any one of [36] to [39], wherein the PD-1 pathway antagonist is at least one or more selected from the group consisting of an anti-PD-1 antibody, an anti-PD-L1 antibody and an anti-PD-L2 antibody.
[41] The use of an azabicyclo compound or a salt thereof according to [40], wherein the anti PD-1-antibody is at least one or more selected from the group consisting of nivolumab and pembrolizumab, and the anti-PD-L1 antibody is at least one or more selected from the group consisting of atezolizumab, durvalumab and avelumab.
[42] The use of an azabicyclo compound or a salt thereof according to any one of [36] to [38], wherein the CTLA-4 pathway antagonist is an anti-CTLA-4 antibody.
[43] The use of an azabicyclo compound or a salt thereof according to [42], wherein the anti-CTLA-4 antibody is at least one or more selected from the group consisting of ipilimumab and tremelimumab.
[44] Use of an azabicyclo compound or a salt thereof for producing an antitumor effect enhancing agent for enhancing the antitumor effect of an immune checkpoint molecule regulator, the azabicyclo compound being represented by the general formula (I):

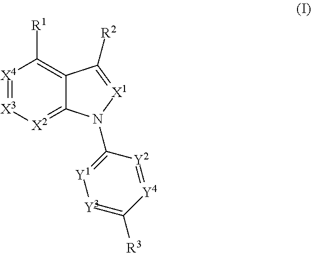

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[45] Use of an azabicyclo compound or a salt thereof for producing an antitumor agent for treating a cancer patient given an immune checkpoint molecule regulator, the azabicyclo compound being represented by the general formula (I):

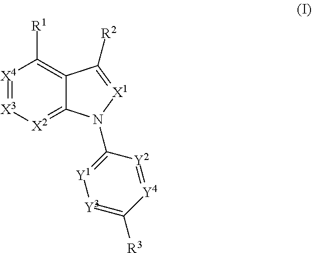

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[46] Use of an immune checkpoint molecule regulator for producing an antitumor agent for treating a cancer patient given an azabicyclo compound or a salt thereof, the azabicyclo compound being a compound represented by the general formula (I):

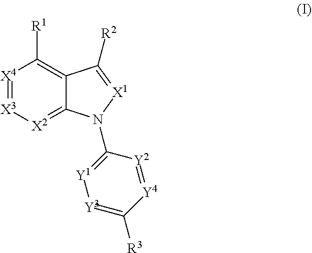

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[47] Use of an azabicyclo compound or a salt thereof and an immune checkpoint molecule regulator for producing an antitumor agent, the azabicyclo compound being represented by the general formula (I):

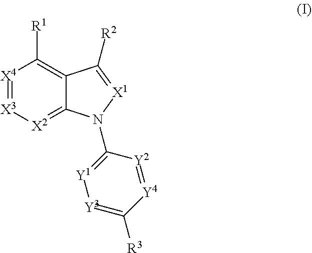

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[48] Use of an azabicyclo compound or a salt thereof for producing an immunostimulant, the azabicyclo compound being represented by the general formula (I):

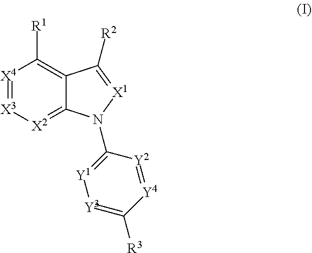

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R⁸ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[49] A method for treating a tumor, comprising administering to a subject in need thereof an azabicyclo compound or a salt thereof and an immune checkpoint molecule regulator in combination, the azabicyclo compound being represented by the general formula (I):

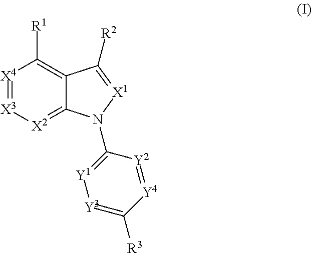

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[50] The method according to [49], wherein the azabicyclo compound is a compound of the general formula (I) in which X¹ is CH or N;
X² is N, and X³ and X⁴ are CH;
Y¹ and Y³ are CH, one or two of Y² and Y⁴ are C—R⁴, and the other is CH;
R¹ is any one of an optionally substituted 1H-imidazol-1-yl group, an optionally substituted pyrazol-4-yl group, an optionally substituted thiophen-3-yl group, an optionally substituted furan-2-yl group, an optionally substituted pyridin-3-yl group, an optionally substituted pyridin-4-yl group, an optionally substituted indol-5-yl group, an optionally substituted 1H-pyrrolo[2,3-b]pyridin-5-yl group, an optionally substituted benzofuran-2-yl group, an optionally substituted quinolin-3-yl group and an optionally substituted 5,6,7,8-tetrahydroquinon-3-yl group;
R² is an alkyl group having 1 to 6 carbon atoms and optionally having a halogen atom, or an alkenyl group having 2 to 6 carbon atoms;
R³ is —CO—R⁵;
R⁴ is an alkyl group having 1 to 6 carbon atoms and optionally having a halogen atom, a mono- or di-(C1-C6 alkyl)amino group or a monocyclic 5- to 7-membered saturated heterocyclic group having 1 or 2 heteroatoms selected from the group consisting of N, S and O, an alkoxy group having 1 to 6 carbon atoms, —N(R⁶)(R⁷), —S—R³ or —CO—R⁹;
R⁵ is an amino group, or a mono- or di-(C1-C6 alkyl)amino group;
R⁶ is a hydrogen atom, or an optionally substituted alkyl group having 1 to 6 carbon atoms;
R⁷ is a hydrogen atom, an optionally substituted alkyl group having 1 to 6 carbon atoms, an optionally substituted cycloalkyl group having 3 to 7 carbon atoms, an optionally substituted aralkyl group having 7 to 12 carbon atoms, an optionally substituted aromatic hydrocarbon group having 6 to 14 carbon atoms, an optionally substituted monocyclic or dicyclic saturated heterocyclic group having 1 to 4 heteroatoms selected from the group consisting of N, S and O, or an optionally substituted monocyclic or dicyclic unsaturated heterocyclic group having 1 to 4 heteroatoms selected from the group consisting of N, S and O, or R⁶ and R⁷ form a 5- to 7-membered saturated heterocyclic group, together with the nitrogen atom to which they are attached;
R⁸ is an optionally substituted cycloalkyl group having 3 to 7 carbon atoms, or an optionally substituted aromatic hydrocarbon group having 6 to 14 carbon atoms; and
R⁹ is a hydrogen atom, a hydroxyl group, an amino group, or a mono- or a di-(C1-C6 alkyl)amino group.
[51] The method according to [49] or [50], wherein the azabicyclo compound is 3-ethyl-4-{3-isopropyl-4-(4-(1-methyl-1H-pyrazol-4-yl)-1H-imidazol-1-yl)-1H-pyrazolo[3,4-b]pyridin-1-yl}benzamide.
[52] The method according to any one of [49] to [51], wherein the immune checkpoint molecule regulator is at least one or more selected from the group consisting of a PD-1 pathway antagonist, an ICOS pathway agonist, a CTLA-4 pathway antagonist and a CD28 pathway agonist.
[53] The method according to any one of [49] to [52], wherein the immune checkpoint molecule regulator is at least one or more selected from the group consisting of a PD-1 pathway antagonist, a CTLA-4 pathway antagonist and a CD28 pathway agonist.
[54] The method according to any one of [49] to [53], wherein the immune checkpoint molecule regulator is at least one or more selected from the group consisting of a PD-1 pathway antagonist and a CTLA-4 pathway antagonist.
[55] The method according to any one of [49] to [54], wherein the immune checkpoint molecule regulator is a PD-1 pathway antagonist.
[56] The method according to any one of [49] to [55], wherein the PD-1 pathway antagonist is at least one or more selected from the group consisting of an anti-PD-1 antibody, an anti-PD-L1 antibody and an anti-PD-L2 antibody.
[57] The method according to [56], wherein the anti PD-1-antibody is at least one or more selected from the group consisting of nivolumab and pembrolizumab, and the anti-PD-L1 antibody is at least one or more selected from the group consisting of atezolizumab, durvalumab and avelumab.
[58] The method according to any one of [52] to [54], wherein the CTLA-4 pathway antagonist is an anti-CTLA-4 antibody.
[59] The method according to [58], wherein the anti-CTLA-4 antibody is at least one or more selected from the group consisting of ipilimumab and tremelimumab.
[60] A method for enhancing the antitumor effect of an immune checkpoint molecule regulator, comprising administering to a subject in need thereof an azabicyclo compound or a salt thereof, the azabicyclo compound being represented by the general formula (I):

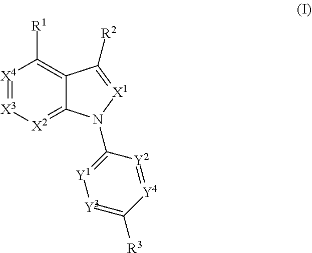

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R⁸ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R⁸ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[61] A method for treating a tumor in a cancer patient given an immune checkpoint molecule regulator, comprising administering to a subject in need thereof an azabicyclo compound or a salt thereof, the azabicyclo compound being represented by the general formula (I):

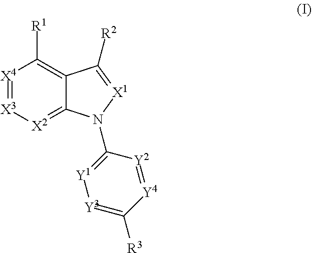

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[62] A method for treating a tumor in a cancer patient given an azabicyclo compound or a salt thereof, comprising administering to a subject in need thereof an immune checkpoint molecule regulator, the azabicyclo compound being a compound represented by the general formula (I):

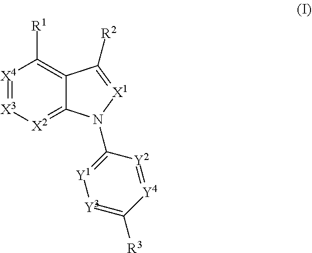

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[63] A method for treating a tumor, comprising administering to a subject in need thereof a combination of an azabicyclo compound or a salt thereof and an immune checkpoint molecule regulator, the azabicyclo compound being represented by the general formula (I):

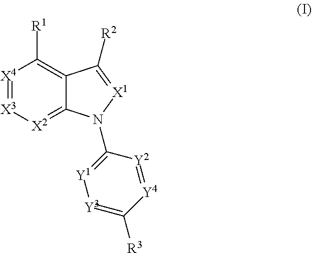

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R⁸ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.
[64] A method for immunostimulation, comprising administering to a subject in need thereof an azabicyclo compound or a salt thereof, the azabicyclo compound being represented by the general formula (I):

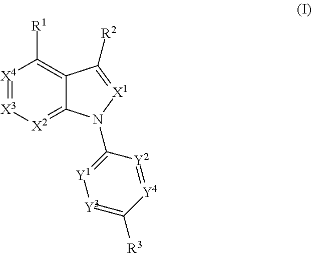

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R⁹ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.

The present invention also relates to the following aspects.
A pharmaceutical composition for preventing and/or treating a tumor, comprising the azabicyclo compound represented by the general formula (I) or a salt thereof and an immune checkpoint molecule regulator.
An azabicyclo compound represented by the general formula (I) or a salt thereof for enhancing the antitumor effect of the immune checkpoint molecule regulator.
Use of an azabicyclo compound represented by the general formula (I) or a salt thereof for enhancing the antitumor effect of the immune checkpoint molecule regulator.
Use of an azabicyclo compound represented by the general formula (I) or a salt thereof for producing an antitumor effect enhancing agent for the immune checkpoint molecule regulator.
A method for preventing and/or treating a tumor, comprising the step of administering a prophylactically and/or therapeutically effective amount of a combination of an azabicyclo compound represented by the general formula (I) or a salt thereof and an immune checkpoint molecule regulator to a patient.
A method for preventing and/or treating a tumor, comprising the step of administering a prophylactically and/or therapeutically effective amount of an azabicyclo compound represented by the general formula (I) or a salt thereof to a cancer patient given an immune checkpoint molecule regulator.
A method for enhancing an antitumor effect, comprising the step of administering a prophylactically and/or therapeutically effective amount of an azabicyclo compound represented by the general formula (I) or a salt thereof to a cancer patient given an immune checkpoint molecule regulator.
A product comprising an azabicyclo compound represented by the general formula (I) or a salt thereof and an immune checkpoint molecule regulator, as a combined preparation to be used simultaneously, sequentially or at intervals in preventing and/or treating a tumor.
An azabicyclo compound represented by the general formula (I) or a salt thereof for immunostimulation.
Use of an azabicyclo compound represented by the general formula (I) or a salt thereof for immunostimulation.
Use of an azabicyclo compound represented by the general formula (I) or a salt thereof for producing an immunostimulant.
A method for immunostimulation, comprising the step of administering an effective amount of an azabicyclo compound represented by the general formula (I) or a salt thereof to a patient in need of the immunostimulation.

### Advantageous Effects of Invention

The antitumor agent of the present invention makes it possible to perform cancer treatment with high antitumor effects (in particular, a tumor reducing effect and a tumor growth delaying effect (life extending effect)) while suppressing development of side-effects. Hence, the life of a cancer patient is extended.

In addition, the immunostimulant of the present invention is expected to be used as new alternatives for treatment of diseases which can be ameliorated by immunostimulation (various infectious diseases, immunodeficiency diseases, etc.).

### BRIEF DESCRIPTION OF DRAWINGS

FIG. 1 shows the effect of a combination of Compound 1 and an antitumor mouse PD-1 antibody in a mouse model which has been transplanted with a mouse bowel cancer cell line MC38.

FIG. 2a shows the ratio of regulatory T-cells in T-cells in tumor infiltrating leucocytes, and FIG. 2b shows the ratio of CD8 positive cells in tumor infiltrating leucocytes.

FIG. 3 shows the effect of a combination of Compound 1 and an antitumor mouse PD-L1 antibody in a mouse model which has been transplanted with a mouse bowel cancer cell line MC38.

FIG. 4 shows the effect of a combination of Compound 1 and an antitumor mouse CTLA-4 antibody in a mouse model which has been transplanted with a mouse bowel cancer cell line MC38.

### DESCRIPTION OF EMBODIMENTS

The present invention relates to an antitumor agent, an antitumor effect enhancing agent, and a kit preparation, comprising an azabicyclo compound represented by the general formula (I) or a salt thereof and an immune checkpoint molecule regulator (in particular, anti-PD-1 antibody, anti-PD-L1 antibody or anti-CTLA-4 antibody), which are administered in combination; use thereof; a method for treating a tumor; a method for enhancing an antitumor effect; and an immunostimulant.

In the present invention, the azabicyclo compound is represented by the following general formula (I):

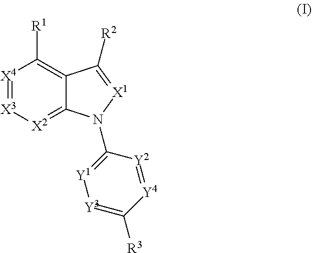

wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R⁸ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.

In the present specification, examples of “substituents” include a halogen atom, a hydroxyl group, a cyano group, a nitro group, an alkyl group, a halogenoalkyl group, a cycloalkyl group, a cycloalkyl-alkyl group, an aralkyl group, a hydroxyalkyl group, an alkenyl group, an alkynyl group, an alkoxy group, a halogenoalkoxy group, an alkoxy-alkyl group, a cycloalkoxy group, a cycloalkyl-alkoxy group, an aralkyloxy group, an aralkyloxy-alkyl group, an alkylthio group, a cycloalkyl-alkylthio group, an amino group, a mono- or dialkylamino group, a cycloalkyl-alkylamino group, an acyl group, an acyloxy group, an oxo group, a carboxyl group, an alkoxycarbonyl group, an aralkyloxycarbonyl group, a carbamoyl group, a saturated or unsaturated heterocyclic group, an aromatic hydrocarbon group and a saturated heterocyclic oxy group. The number of the substituents, if any, is typically 1 to 3.

Examples of the halogen atom included in the substituents include chlorine, bromine, fluorine and iodine atoms.

The alkyl or halogenoalkyl group included in the substituents preferably refers to a linear or branched alkyl group having 1 to 6 carbon atoms, or a group in which one or all hydrogen atoms in such an alkyl group are substituted by the halogen atom exemplified above. Examples thereof include: alkyl groups such as methyl, ethyl, n-propyl, isopropyl, n-butyl, isobutyl, sec-butyl, tert-butyl, pentyl and hexyl groups; and halogenoalkyl groups such as a trifluoromethyl group.

The cycloalkyl group included in the substituents is preferably a cycloalkyl group having 3 to 7 carbon atoms. Examples thereof include cyclopropyl, cyclobutyl, cyclopentyl, cyclohexyl and cycloheptyl groups.

The cycloalkyl-alkyl group included in the substituents is preferably an alkyl group having 1 to 6 carbon atoms which is substituted by cycloalkyl having 3 to 7 carbon atoms. Examples thereof include cyclopropylmethyl, cyclopropylethyl, cyclobutylmethyl, cyclopentylmethyl and cyclohexylmethyl groups.

The aralkyl group included in the substituents preferably refers to a linear or branched alkyl group having 1 to 6 carbon atoms which is substituted by an aromatic hydrocarbon group having 6 to 14 carbon atoms. Examples thereof include benzyl, phenylethyl, phenylpropyl, naphthylmethyl and naphthylethyl groups.

The hydroxyalkyl group included in the substituents preferably refers to the linear or branched alkyl group having 1 to 6 carbon atoms exemplified above which has a hydroxy group. Examples thereof include hydroxymethyl and hydroxyethyl groups.

The alkenyl group included in the substituents preferably refers to an alkenyl group having 2 to 6 carbon atoms which contains a carbon-carbon double bond. Examples thereof include vinyl, allyl, methylvinyl, propenyl, butenyl, pentenyl and hexenyl groups.

The alkynyl group included in the substituents preferably refers to an alkynyl group having 2 to 6 carbon atoms which contains a carbon-carbon triple bond. Examples thereof include ethynyl and propargyl groups.

The alkoxy or halogenoalkoxy group included in the substituents preferably refers to a linear or branched alkoxy group having 1 to 6 carbon atoms, or a group in which such an alkoxy group is substituted by the halogen atom exemplified above. Examples thereof include methoxy, ethoxy, n-propoxy, isopropoxy, 1-methylpropoxy, n-butoxy, isobutoxy, tert-butoxy, 2-methyl-butoxy, neopentyloxy, pentan-2-yloxy, fluoromethoxy, difluoromethoxy, trifluoromethoxy, 1,1-difluoroethoxy, 2,2-difluoroethoxy, 2,2,2-trifluoroethoxy, 1,1,2,2-tetrafluoroethoxy, perfluoroethoxy, 3-fluoro-2-(fluoromethyl)-propoxy, 1,3-difluoropropan-2-yloxy and 2,2,3,3,3-pentafluoro-1-propoxy groups.

The cycloalkoxy group included in the substituents is preferably a cycloalkoxy group having 3 to 7 carbon atoms. Examples thereof include cyclopropoxy, cyclobutoxy, cyclopentyloxy, cyclohexyloxy and cycloheptyloxy groups.

The alkoxy-alkyl group included in the substituents preferably refers to the alkyl group having 1 to 6 carbon atoms exemplified above which is substituted by the linear or branched alkoxy group having 1 to 6 carbon atoms exemplified above. Examples thereof include methoxymethyl and ethoxymethyl groups.

The cycloalkyl-alkoxy group included in the substituents is preferably an alkoxy group having 1 to 6 carbon atoms which is substituted by cycloalkyl having 3 to 7 carbon atoms. Examples thereof include cyclopropylmethoxy, cyclopropylethoxy, cyclobutylmethoxy, cyclopentylmethoxy and cyclohexylmethoxy groups.

The aralkyloxy group included in the substituents preferably refers to an oxy group which has the aralkyl group exemplified above. Examples thereof include benzyloxy, phenethyloxy, phenylpropyloxy, naphthylmethyloxy and naphthylethyloxy groups.

The aralkyloxy-alkyl group included in the substituents preferably refers to the linear or branched alkyl group having 1 to 6 carbon atoms exemplified above which has the aralkyloxy group exemplified above. Examples thereof include benzyloxymethyl and benzyloxyethyl groups.

The alkylthio group included in the substituents is preferably a (C1-C6) alkylthio group which refers to a linear or branched alkylthio group having 1 to 6 carbon atoms. Examples thereof include methylthio, ethylthio, n-propylthio, isopropylthio, n-butylthio, isobutylthio, sec-butylthio, tert-butylthio, pentylthio and hexylthio groups.

The cycloalkyl-alkylthio group included in the substituents is preferably an alkylthio group having 1 to 6 carbon atoms which is substituted by cycloalkyl having 3 to 7 carbon atoms. Examples thereof include cyclopropylmethylthio, cyclopropylethylthio, cyclobutylmethylthio, cyclopentylmethylthio and cyclohexylmethylthio groups.

The mono- or dialkylamino group included in the substituents is a mono- or di-(C1-C6 alkyl)amino group which refers to an amino group which is monosubstituted or disubstituted by the linear or branched alkyl group having 1 to 6 carbon atoms exemplified above. Examples thereof include methylamino, dimethylamino, ethylamino, diethylamino and methylethylamino groups.

The cycloalkyl-alkylamino group included in the substituents refers to an alkylamino group which is substituted by the cycloalkyl group exemplified above. Examples thereof include cyclopropylmethylamino, cyclobutylmethylamino and cyclopentylmethylamino groups.

Examples of the acyl group included in the substituents include: linear or branched acyl groups having 1 to 6 carbon atoms, such as formyl, acetyl, propionyl, n-butyryl, isobutyryl, valeryl, isovaleryl and pivaloyl groups; and a benzoyl group.

Examples of the acyloxy group included in the substituents include: linear or branched acyloxy groups having 1 to 6 carbon atoms, such as formyloxy, acetoxy, propionyloxy, n-butyryloxy, isobutyryloxy, valeryloxy, isovaleryloxy and pivaloyloxy groups; a benzoyloxy group; and amino acid-derived acyloxy groups such as glycyloxy, alanyloxy and leucyloxy groups.

The alkoxycarbonyl group included in the substituents refers to a carbonyl group which is substituted by the alkoxy group exemplified above. Examples thereof include methoxycarbonyl, ethoxycarbonyl, n-propoxycarbonyl, isopropoxycarbonyl, 1-methylpropoxycarbonyl, n-butoxycarbonyl, isobutoxycarbonyl, tert-butoxycarbonyl, 2-methyl-butoxycarbonyl, neopentyloxycarbonyl and pentan-2-yloxycarbonyl groups.

The aralkyloxycarbonyl group included in the substituents preferably refers to a carbonyl group which is substituted by the aralkyloxy group exemplified above. Examples thereof include benzyloxycarbonyl, phenethyloxycarbonyl, phenylpropyloxycarbonyl, naphthylmethyloxycarbonyl and naphthylethyloxycarbonyl groups.

Examples of the carbamoyl group included in the substituents include —CONH₂, (mono- or dialkyl)carbamoyl, (mono- or diaryl)carbamoyl, (N-alkyl-N-aryl)carbamoyl, pyrrolidinocarbamoyl, piperidinocarbamoyl, piperazinocarbamoyl and morpholinocarbamoyl groups.

The saturated or unsaturated heterocyclic group included in the substituents refers to a monocyclic or bicyclic saturated or 5- to 10-membered unsaturated heterocyclic group preferably having 1 to 4 of any heteroatom of N, S and O. Examples thereof include pyrrolidinyl, piperidinyl, piperazinyl, hexamethyleneimino, morpholino, thiomorpholino, homopiperazinyl, tetrahydrofuranyl, tetrahydropyranyl, imidazolyl, thienyl, furyl, pyrrolyl, oxazolyl, isoxazolyl, thiazolyl, isothiazolyl, pyrazolyl, triazolyl, tetrazolyl, pyridyl, pyrazyl, pyrimidinyl, pyridazinyl, indolyl, isoindolyl, indazolyl, methylenedioxyphenyl, ethylenedioxyphenyl, benzofuranyl, dihydrobenzofuranyl, benzoimidazolyl, benzoxazolyl, benzothiazolyl, purinyl, quinolyl, isoquinolyl, quinazolinyl and quinoxalyl groups.

The aromatic hydrocarbon group included in the substituents preferably refers to an aromatic hydrocarbon group having 6 to 14 carbon atoms. Examples thereof include phenyl and naphthyl groups.

The saturated heterocyclic oxy group included in the substituents refers to a monocyclic 5- to 7-membered saturated heterocyclic group having one or two of any heteroatom of N, S and 0, for example, an oxy group which has a pyrrolidinyl, piperidinyl, piperazinyl, hexamethyleneimino, morpholino, thiomorpholino, homopiperazinyl group or the like. Examples thereof include tetrahydrofuranyloxy and tetrahydropyranyloxy groups.

In the general formula (I), X¹ represents CH or N. Moreover, in the general formula (I), any one of X², X³ and X⁴ represents N, and the others represent CH. Based on these definitions of X¹ to X⁴, examples of the azabicyclo skeleton in the general formula (I) include the following structures:

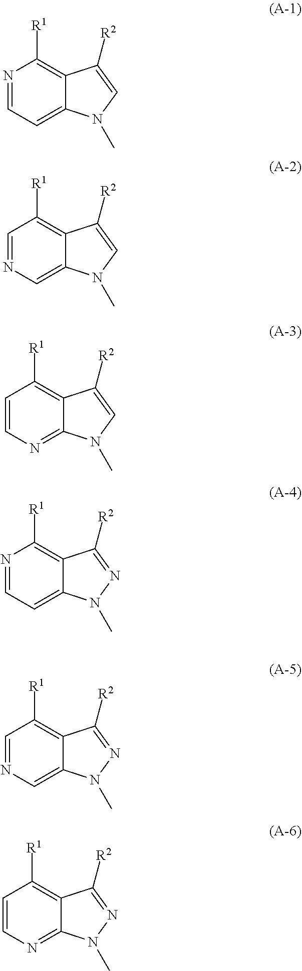

wherein R¹ and R² are as defined above.

Of these skeletons, (A-3) and (A-6) are particularly preferable.

In the general formula (I), the “monocyclic or bicyclic unsaturated heterocyclic group having 1 to 4 heteroatoms selected from N, S and O” in the “optionally substituted monocyclic or bicyclic unsaturated heterocyclic group having 1 to 4 heteroatoms selected from N, S and O”, represented by R¹ is preferably a monocyclic or bicyclic 5- to 10-membered unsaturated heterocyclic group having 1 to 3 heteroatoms selected from N, S and O, more preferably a monocyclic 5- to 6-membered unsaturated heterocyclic group having 1 to 3 heteroatoms selected from N, S and O, or a bicyclic 9- to 10-membered unsaturated heterocyclic group having 1 to 3 heteroatoms selected from N, S and O.

The heterocyclic group is preferably a group having imidazole, pyrazole, thiophene, furan, pyrrole, oxazole, isoxazole, thiazole, isothiazole, triazole, tetrazole, pyridine, pyrazine, pyrimidine, pyridazine, indole, isoindole, pyrrolopyridine, indazole, methylenedioxyphenyl, ethylenedioxyphenyl, benzofuran, dihydrobenzofuran, benzimidazole, benzoxazole, benzothiazole, purine, quinoline, tetrahydroquinoline, isoquinoline, quinazoline or quinoxaline, more preferably a group having imidazole, pyrazole, thiophene, furan, pyridine, indole, pyrrolopyridine, benzofuran, quinoline or tetrahydroquinoline, particularly preferably a group having imidazole, pyridine or quinoline.

Specific examples thereof include 1H-imidazol-1-yl, 1H-imidazol-2-yl, 1H-imidazol-4-yl, 1H-pyrazol-1-yl, 1H-pyrazol-3-yl, 1H-pyrazol-4-yl, thiophen-2-yl, thiophen-3-yl, furan-2-yl, furan-3-yl, pyrrol-1-yl, pyrrol-2-yl, pyrrol-3-yl, oxazol-2-yl, oxazol-4-yl, oxazol-5-yl, isoxazol-3-yl, isoxazol-4-yl, isoxazol-5-yl, thiazol-2-yl, thiazol-3-yl, thiazol-4-yl, thiazol-5-yl, isothiazol-2-yl, isothiazol-4-yl, isothiazol-5-yl, pyrazol-1-yl, pyrazol-3-yl, pyrazol-4-yl, 1,2,3-triazol-1-yl, 1,2,3-triazol-4-yl, 1,2,4-triazol-1-yl, 1,2,4-triazol-3-yl, 1,2,4-triazol-4-yl, tetrazol-1-yl, tetrazol-5-yl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyrimidin-6-yl, pyridazin-3-yl, pyridazin-4-yl, indol-1-yl, indol-2-yl, indol-3-yl, indol-4-yl, indol-5-yl, indol-6-yl, indol-7-yl, isoindol-1-yl, isoindol-2-yl, isoindol-4-yl, isoindol-5-yl, 1H-pyrrolo[2,3-b]pyridin-1-yl, 1H-pyrrolo[2,3-b]pyridin-2-yl, 1H-pyrrolo[2,3-b]pyridin-3-yl, 1H-pyrrolo[2,3-b]pyridin-4-yl, 1H-pyrrolo[2,3-b]pyridin-5-yl, 1H-pyrrolo[2,3-b]pyridin-6-yl, 1H-indazol-1-yl, 1H-indazol-3-yl, 1H-indazol-4-yl, 1H-indazol-5-yl, 1H-indazol-6-yl, 1H-indazol-7-yl, methylenedioxyphenyl, ethylenedioxyphenyl, benzofuran-2-yl, benzofuran-3-yl, benzofuran-4-yl, benzofuran-5-yl, benzofuran-6-yl, benzofuran-7-yl, 2,3-dihydrobenzofuran-2-yl, 2,3-dihydrobenzofuran-3-yl, benzimidazol-1-yl, benzimidazol-2-yl, benzimidazol-4-yl, benzimidazol-5-yl, benzoxazol-2-yl, benzoxazol-4-yl, benzoxazol-5-yl, benzothiazol-2-yl, benzothiazol-4-yl, benzothiazol-5-yl, purin-2-yl, purin-6-yl, purin-7-yl, purin-8-yl, quinolin-2-yl, quinolin-3-yl, quinolin-4-yl, quinolin-5-yl, quinolin-6-yl, quinolin-7-yl, quinolin-8-yl, 5,6,7,8-tetrahydroquinolin-2-yl, 5,6,7,8-tetrahydroquinolin-3-yl, 5,6,7,8-tetrahydroquinolin-4-yl, isoquinolin-1-yl, isoquinolin-3-yl, isoquinolin-4-yl, isoquinolin-5-yl, isoquinolin-6-yl, isoquinolin-7-yl, isoquinolin-8-yl, quinazolin-4-yl, quinoxalin-2-yl, quinoxalin-5-yl and quinoxalin-6-yl groups. The heterocyclic group is preferably a 1H-imidazol-1-yl, pyrazol-4-yl, thiophen-3-yl, furan-2-yl, pyridin-3-yl, pyridin-4-yl, indol-5-yl, 1H-pyrrolo[2,3-b]pyridin-5-yl, benzofuran-2-yl, quinolin-3-yl or 5,6,7,8-tetrahydroquinolin-3-yl group, more preferably a 1H-imidazol-1-yl, pyridin-3-yl, pyridin-4-yl, indol-5-yl, 1H-pyrrolo[2,3-b]pyridin-5-yl, benzofuran-2-yl, quinolin-3-yl or 5,6,7,8-tetrahydroquinolin-3-yl group, particularly preferably a 1H-imidazol-1-yl, pyridin-3-yl or quinolin-3-yl group.

In the general formula (I), examples of the “substituent(s)” in the unsaturated heterocyclic group represented by R¹ include the substituents exemplified above. The substituent(s) are preferably 1 to 3 substituents selected from an alkyl group, an alkoxy group, an alkoxy-alkyl group, an aralkyl group, an aralkyloxy-alkyl group, a halogen atom, a halogenoalkyl group, an acyl group, an optionally substituted saturated or unsaturated heterocyclic group and an optionally substituted aromatic hydrocarbon group, more preferably 1 to 3 substituents selected from: an alkyl group; an alkoxy group; an unsaturated heterocyclic group optionally having an alkyl group, a halogenoalkyl group, an aralkyl group or a hydroxyalkyl group; and an aromatic hydrocarbon group optionally having an alkyl group, an alkoxy group or a carbamoyl group. In this context, examples of the unsaturated heterocyclic group which may be substituted on the unsaturated heterocyclic ring represented by R¹ include pyrazole, imidazole, pyridine, pyrimidine, furan and thiophene. Moreover, examples of the aromatic hydrocarbon group include phenyl and naphthyl.

Specific examples of the “substituent(s)” in the unsaturated heterocyclic group represented by R¹ can include methyl, ethyl, n-propyl, isopropyl, n-butyl, isobutyl, sec-butyl, tert-butyl, methoxy, ethoxy, n-propoxy, isopropoxy, 1-methylpropoxy, n-butoxy, isobutoxy, tert-butoxy, 1H-pyrazol-4-yl, 1-methyl-1H-pyrazol-4-yl, 1-ethyl-1H-pyrazol-4-yl, 1-isopropyl-1H-pyrazol-4-yl, 1-benzyl-1H-pyrazol-4-yl, 1-(difluoromethyl)-1H-pyrazol-4-yl, 1-(hydroxyethyl)-1H-pyrazol-4-yl, 1H-imidazol-1-yl, pyridin-3-yl, pyridin-4-yl, pyrimidin-5-yl, furan-2-yl, furan-3-yl, thiophen-3-yl, phenyl, 4-methoxyphenyl, 4-carbamoylphenyl, 4-isopropylcarbamoylphenyl and 4-dimethylcarbamoylphenyl groups.

Specific examples of preferable R¹ include 1H-imidazol-1-yl, 4-phenyl-1H-imidazol-1-yl, 4-(4-carbamoylphenyl)-1H-imidazol-1-yl, 4-(4-methoxyphenyl)-1H-imidazol-1-yl, 4-(thiophen-3-yl)-1H-imidazol-1-yl, 4-(pyridin-3-yl)-1H-imidazol-1-yl, 4-(pyridin-4-yl)-1H-imidazol-1-yl, 5-methyl-4-(pyridin-3-yl)-1H-imidazol-1-yl, 4-(pyrimidin-5-yl)-1H-imidazol-1-yl, 4-(furan-2-yl)-1H-imidazol-1-yl, 4-(furan-3-yl)-1H-imidazol-1-yl, 4-(1H-pyrazol-4-yl)-1H-imidazol-1-yl, 4-(1-methyl-1H-pyrazol-4-yl)-1H-imidazol-1-yl, 4-(1-ethyl-1H-pyrazol-4-yl)-1H-imidazol-1-yl, 4-(1-isopropyl-1H-pyrazol-4-yl)-1H-imidazol-1-yl, 4-(1-hydroxymethyl)-(1H-pyrazol-4-yl)-1H-imidazol-1-yl, 4-(1-(difluoromethyl)-1H-pyrazol-4-yl)-1H-imidazol-1-yl, 4-(1-(hydroxyethyl)-1H-pyrazol-4-yl)-1H-imidazol-1-yl, 4-(1-(hydroxymethyl)-1H-pyrazol-4-yl)-1H-imidazol-1-yl, 4-(1-benzyl-1H-pyrazol-4-yl)-1H-imidazol-1-yl, 4-(1-(benzyloxyethyl)-1H-pyrazol-4-yl)-1H-imidazol-1-yl, 1¹H-1,4′-biimidazol-1′-yl, pyridin-3-yl, pyridin-4-yl, 5-methoxypyridin-3-yl, 6-methoxypyridin-3-yl, 1-benzyl-1H-pyrazol-4-yl, 1-methyl-1H-indol-5-yl, 1H-pyrrolo[2,3-b]pyridin-5-yl, 1-methyl-1H-pyrrolo[2,3-b]pyridin-5-yl, 1-methoxymethyl-1H-pyrrolo[2,3-b]pyridin-5-yl, 5,6,7,8-tetrahydroquinolin-3-yl, quinolin-3-yl, thiophen-3-yl, furan-2-yl and benzofuran-2-yl groups. R¹ is more preferably a 1H-imidazol-1-yl, 4-(pyridin-3-yl)-1H-imidazol-1-yl, 4-(pyridin-4-yl)-1H-imidazol-1-yl, 4-(1H-pyrazol-4-yl)-1H-imidazol-1-yl, 4-(1-methyl-1H-pyrazol-4-yl)-1H-imidazol-1-yl, 4-(1-ethyl-1H-pyrazol-4-yl)-1H-imidazol-1-yl, 4-(1-isopropyl-1H-pyrazol-4-yl)-1H-imidazol-1-yl, 4-(1-benzyl-1H-pyrazol-4-yl)-1H-imidazol-1-yl or quinolin-3-yl group, particularly preferably a 4-(1-methyl-1H-pyrazol-4-yl)-1H-imidazol-1-yl, 4-(pyridin-3-yl)-1H-imidazol-1-yl or quinolin-3-yl group.

In the general formula (I), the “alkyl group having 1 to 6 carbon atoms” in the “optionally substituted alkyl group having 1 to 6 carbon atoms” represented by R² refers to a linear or branched alkyl group having 1 to 6 carbon atoms, for example, a methyl, ethyl, n-propyl, isopropyl, n-butyl, isobutyl, sec-butyl, tert-butyl, pentyl or hexyl group, and is preferably a methyl, ethyl, n-propyl or isopropyl group.

Examples of the “substituent(s)” in the “optionally substituted alkyl group having 1 to 6 carbon atoms” represented by R² include the substituents exemplified above. Of them, the substituent(s) are preferably a halogen atom.

The halogen atom-substituted alkyl group is preferably a halogenoalkyl group having 1 to 6 carbon atoms, more preferably a trifluoromethyl group.

The “alkenyl group having 2 to 6 carbon atoms” in the “optionally substituted alkenyl group having 2 to 6 carbon atoms”, represented by R² is the aforementioned alkenyl group having 2 to 6 carbon atoms, preferably a vinyl group. Examples of the “substituent” in the “optionally substituted alkenyl group having 2 to 6 carbon atoms” include the aforementioned substituents.

R² is more preferably an optionally substituted alkyl group having 1 to 6 carbon atoms, or an optionally substituted alkenyl group having 2 to 6 carbon atoms, even more preferably an alkyl group having 1 to 6 carbon atoms which optionally has a halogen atom, or an alkenyl group having 2 to 6 carbon atoms, particularly preferably an alkyl group having 1 to 4 carbon atoms which optionally has a halogen atom.

Any one or two of Y¹, Y², Y³ and Y⁴ represent C—R⁴, and the others are the same or different and represent CH or N. Of them, preferably, any one or two of Y¹, Y², Y³ and Y⁴ are C—R⁴, and the others are CH. More preferably, Y¹ and Y³ are CH, any one or two of Y² and Y⁴ are C—R⁴, and the other is CH. These preferable aspects are represented by the following structural formulas:

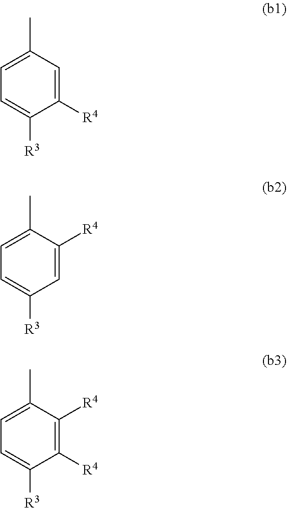

wherein R³ and R⁴ are as defined above.

Of them, (b1) and (b2) are particularly preferable.

In the general formula (I), R³ represents a cyano group or —CO—R⁵. Of them, —CO—R⁵ is particularly preferable.

In the general formula (I), R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹. Of them, R⁴ is preferably a halogen atom, an alkyl group having 1 to 6 carbon atoms which optionally has a mono- or di-(C1-C6 alkyl)amino group or a monocyclic 5- to 7-membered saturated heterocyclic group having one or two of any heteroatom of N, S and O, an alkoxy group having 1 to 6 carbon atoms, —N(R⁶)(R⁷), —S—R⁸ or —CO—R⁹, more preferably a halogen atom, an alkyl group having 1 to 6 carbon atoms or —N(R⁶)(R⁷).

In the general formula (I), the “halogen atom” represented by R⁴ refers to the halogen atom exemplified above and is preferably a chlorine atom.

In the general formula (I), the “alkyl group having 1 to 6 carbon atoms” in the “optionally substituted alkyl group having 1 to 6 carbon atoms” represented by R⁴ refers to the alkyl group having 1 to 6 carbon atoms exemplified above and is preferably a methyl, ethyl, n-propyl or isopropyl group.

Examples of the “substituent(s)” in the “optionally substituted alkyl group having 1 to 6 carbon atoms” represented by R⁴ include the substituents exemplified above. The “substituent(s)” are preferably mono- or di-(C1-C6 alkyl)amino groups (e.g., ethylamino and dimethylamino groups) or monocyclic 5- to 7-membered saturated heterocyclic groups having one or two of any heteroatom of N, S and O (e.g., pyrrolidyl and morpholino groups).

In the general formula (I), the “alkenyl group having 2 to 6 carbon atoms” represented by R⁴ refers to the alkenyl group having 2 to 6 carbon atoms exemplified above and is preferably a vinyl or prop-1-en-2-yl group.

In the general formula (I), the “alkoxy group having 1 to 6 carbon atoms” represented by R⁴ refers to the alkoxy group having 1 to 6 carbon atoms exemplified above and is preferably a methoxy group.

In the general formula (I), the “mono- or di-alkylamino group” in the “optionally substituted mono- or di-alkylamino group” represented by R⁵ refers to the mono- or dialkylamino group exemplified above and is preferably a mono- or di-(C1-C6 alkyl)amino group.

Examples of the “substituent(s)” in the “optionally substituted mono- or di-alkylamino group” represented by R⁵ include the substituents exemplified above.

R⁵ is more preferably an amino, hydroxylamino or mono- or di-(C1-C6 alkyl)amino group, particularly preferably an amino group.

In the general formula (I), the “alkyl group having 1 to 6 carbon atoms” in the “optionally substituted alkyl group having 1 to 6 carbon atoms” represented by R⁶ or R⁷ refers to the alkyl group having 1 to 6 carbon atoms exemplified above and is preferably an ethyl, n-propyl, n-butyl, isobutyl, sec-butyl or pentyl group.

Examples of the “substituent(s)” in the “optionally substituted alkyl group having 1 to 6 carbon atoms” represented by R⁶ or R⁷ include the substituents exemplified above. The “substituent(s)” are preferably a hydroxyl group, cycloalkyl groups having 3 to 7 carbon atoms (e.g., a cyclohexyl group), saturated heterocyclic groups (e.g., pyrrolidyl and morpholino groups), unsaturated heterocyclic groups (e.g., a pyridyl group), mono- or di-(C1-C6 alkyl)amino groups (e.g., ethylamino and dimethylamino groups), (C1-C6 alkyl)thio groups (e.g., a methylthio group), or alkoxy groups having 1 to 6 carbon atoms which optionally has a hydroxyl group.

In the general formula (I), the “halogenoalkyl group having 1 to 6 carbon atoms” represented by R⁶ or R⁷ refers to the halogenoalkyl group having 1 to 6 carbon atoms exemplified above and is preferably a 2,2-difluoroethyl or 2,2,2-trifluoroethyl group.

In the general formula (I), examples of the “cycloalkyl group having 3 to 7 carbon atoms” in the “optionally substituted cycloalkyl group having 3 to 7 carbon atoms” represented by R⁶ or R⁷ include cyclopropyl, cyclobutyl, cyclopentyl, cyclohexyl and cycloheptyl groups. The cycloalkyl group having 3 to 7 carbon atoms is preferably a cyclopropyl, cyclopentyl or cyclohexyl group.

Examples of the “substituent(s)” in the “optionally substituted cycloalkyl group having 3 to 7 carbon atoms” represented by R⁶ or R⁷ include the substituents exemplified above. The substituent(s) are preferably hydroxyl, amino, amino acid-derived acyloxy, alkanoylamino or alkylsulfonylamino groups, or the like.

In the general formula (I), the “aralkyl group” in the “optionally substituted aralkyl group” represented by R⁶ or R⁷ refers to the aralkyl group exemplified above and is preferably an aralkyl group having 7 to 12 carbon atoms, specifically, a benzyl group.

Examples of the “substituent(s)” in the “optionally substituted aralkyl group” represented by R⁶ or R⁷ include the substituents exemplified above. Specific examples of the substituent(s) include saturated heterocyclic groups such as a pyrrolidinyl group.

In the general formula (I), the “aromatic hydrocarbon group” in the “optionally substituted aromatic hydrocarbon group” represented by R⁶ or R⁷ refers to the aromatic hydrocarbon group having 6 to 14 carbon atoms exemplified above and is preferably a phenyl group.

Examples of the “substituent(s)” in the “optionally substituted aromatic hydrocarbon group” represented by R⁶ or R⁷ include the substituents exemplified above. The substituent(s) are preferably halogen atoms, alkylthio groups (e.g., a methylthio group), saturated heterocyclic groups (e.g., a morpholino group), or substituted carbamoyl groups (e.g., a pyrrolidine-carbonyl group).

In the general formula (I), the “saturated heterocyclic group” in the “optionally substituted saturated heterocyclic group” represented by R⁶ or R⁷ refers to the saturated heterocyclic group exemplified above and is preferably a piperidinyl or tetrahydropyranyl group.

Examples of the “substituent(s)” in the “optionally substituted saturated heterocyclic group” represented by R⁶ or R⁷ include the substituents exemplified above. The substituent(s) are preferably alkyl groups having 1 to 6 carbon atoms (e.g., a methyl group), acyl groups (e.g., an acetyl group), carbonyl groups having a saturated heterocyclic group (e.g., a 2,6-dihydroxypyrimidinyl-4-carbonyl group), or aminoalkylcarbonyl groups (e.g., a 2-aminoacetyl group).

In the general formula (I), the “unsaturated heterocyclic group” in the “optionally substituted unsaturated heterocyclic group” represented by R⁶ or R⁷ refers to the unsaturated heterocyclic group exemplified above and is preferably a pyridyl or oxazolyl group.

Examples of the “substituent(s)” in the “optionally substituted unsaturated heterocyclic group” represented by R⁶ or R⁷ include the substituents exemplified above.

In the general formula (I), the “saturated heterocyclic group” which is optionally formed by R⁶ and R⁷ together with the nitrogen atom to which they are attached refers to a monocyclic or bicyclic saturated heterocyclic group preferably having 1 to 4 of any atom of oxygen, nitrogen and sulfur atoms, for example, a pyrrolidinyl, piperidinyl, piperazinyl, hexamethyleneimino, morpholino, thiomorpholino, homopiperazinyl, tetrahydrofuranyl or tetrahydropyranyl group.

In the general formula (I), it is preferred for the combination of R⁶ and R⁷ that R⁶ should be a hydrogen atom or an optionally substituted alkyl group having 1 to 6 carbon atoms, and R⁷ should represent a hydrogen atom, an optionally substituted alkyl group having 1 to 6 carbon atoms, an optionally substituted cycloalkyl group having 3 to 7 carbon atoms, an optionally substituted aralkyl group having 7 to 12 carbon atoms, an optionally substituted aromatic hydrocarbon group having 6 to 14 carbon atoms, an optionally substituted monocyclic or bicyclic saturated heterocyclic group having 1 to 4 heteroatoms selected from N, S and O, or an optionally substituted monocyclic or bicyclic unsaturated heterocyclic group having 1 to 4 heteroatoms selected from N, S and O, or R⁶ and R⁷ should optionally form a 5- to 7-membered saturated heterocyclic group, together with the nitrogen atom to which they are attached. More preferably, R⁶ is a hydrogen atom, and R⁷ is a hydrogen atom, an optionally substituted alkyl group having 1 to 6 carbon atoms, an optionally substituted cycloalkyl group having 3 to 7 carbon atoms, or an optionally substituted monocyclic or bicyclic saturated heterocyclic group having 1 to 4 heteroatoms selected from N, S and O. Particularly preferably, R⁶ is a hydrogen atom, and R⁷ is an optionally substituted alkyl group having 1 to 6 carbon atoms or an optionally substituted cycloalkyl group having 3 to 7 carbon atoms.

In the general formula (I), the “cycloalkyl group having 3 to 7 carbon atoms” in the “optionally substituted cycloalkyl group having 3 to 7 carbon atoms” represented by R⁶ refers to the cycloalkyl group having 3 to 7 carbon atoms exemplified above and is preferably a cyclohexyl group. Examples of the “substituent(s)” in the “optionally substituted cycloalkyl group having 3 to 7 carbon atoms” represented by R⁶ include the substituents exemplified above. The substituent(s) are preferably a hydroxyl group.

In the general formula (I), the “aromatic hydrocarbon group” in the “optionally substituted aromatic hydrocarbon group” represented by R⁶ refers to the aromatic hydrocarbon group having 6 to 14 carbon atoms exemplified above and is preferably a phenyl group.

Examples of the “substituent(s)” in the “optionally substituted aromatic hydrocarbon group” represented by R⁸ include the substituents exemplified above. The substituent(s) are preferably a hydroxyl group.

R³ is preferably an optionally substituted cycloalkyl group having 3 to 7 carbon atoms, or an optionally substituted aromatic hydrocarbon group having 6 to 14 carbon atoms.

In the general formula (I), the “mono- or di-alkylamino group” in the “optionally substituted mono- or di-alkylamino group” represented by R⁹ refers to the mono- or dialkylamino group exemplified above and is preferably a mono- or di-(C1-C6 alkyl)amino group.

Examples of the “substituent(s)” in the “optionally substituted mono- or di-alkylamino group” represented by R⁹ include the substituents exemplified above.

R⁹ is preferably a hydrogen atom, a hydroxyl group, an amino group or a mono- or di-(C1-C6 alkyl)amino group, particularly preferably a hydrogen atom.

The preferred azabicyclo compound of the present invention is a compound of the general formula (I) in which X¹ is CH or N; X² is N, and X³ and X⁴ are CH; Y¹ and Y³ are CH, one or two of Y² and Y⁴ are C—R⁴, and the other is CH; R¹ is any one of an optionally substituted 1H-imidazol-1-yl group, an optionally substituted pyrazol-4-yl group, an optionally substituted thiophen-3-yl group, an optionally substituted furan-2-yl group, an optionally substituted pyridin-3-yl group, an optionally substituted pyridin-4-yl group, an optionally substituted indol-5-yl group, an optionally substituted 1H-pyrrolo[2,3-b]pyridin-5-yl group, an optionally substituted benzofuran-2-yl group, an optionally substituted quinolin-3-yl group and an optionally substituted 5,6,7,8-tetrahydroquinon-3-yl group; R² is an alkyl group having 1 to 6 carbon atoms and optionally having a halogen atom, or an alkenyl group having 2 to 6 carbon atoms; R³ is —CO—R⁵; R⁴ is an alkyl group having 1 to 6 carbon atoms and optionally having a halogen atom, a mono- or di-(C1-C6 alkyl)amino group or a monocyclic 5- to 7-membered saturated heterocyclic group having 1 or 2 heteroatoms selected from the group consisting of N, S and O, an alkoxy group having 1 to 6 carbon atoms, —N(R⁶)(R⁷), —S—R³ or —CO—R⁹; R⁵ is an amino group, or a mono- or di-(C1-C6 alkyl)amino group; R⁶ is a hydrogen atom, or an optionally substituted alkyl group having 1 to 6 carbon atoms; R⁷ is a hydrogen atom, an optionally substituted alkyl group having 1 to 6 carbon atoms, an optionally substituted cycloalkyl group having 3 to 7 carbon atoms, an optionally substituted aralkyl group having 7 to 12 carbon atoms, an optionally substituted aromatic hydrocarbon group having 6 to 14 carbon atoms, an optionally substituted monocyclic or dicyclic saturated heterocyclic group having 1 to 4 heteroatoms selected from the group consisting of N, S and O, or an optionally substituted monocyclic or dicyclic unsaturated heterocyclic group having 1 to 4 heteroatoms selected from the group consisting of N, S and O, or R⁶ and R⁷ form a 5- to 7-membered saturated heterocyclic group, together with the nitrogen atom to which they are attached; R⁸ is an optionally substituted cycloalkyl group having 3 to 7 carbon atoms, or an optionally substituted aromatic hydrocarbon group having 6 to 14 carbon atoms; and R⁹ is a hydrogen atom, a hydroxyl group, an amino group, or a mono- or a di-(C1-C6 alkyl)amino group.

The more specific azabicyclo compound is 3-ethyl-4-{3-isopropyl-4-(4-(1-methyl-1H-pyrazol-4-yl)-1H-imidazol-1-yl)-1H-pyrazolo[3,4-b]pyridin-1-yl}benzamide (hereinafter, referred to as Compound 1).

The salt of the azabicyclo compound represented by the general formula (I) according to the present invention is not particularly limited as long as it is a pharmaceutically acceptable salt, and examples thereof include acid addition salts with inorganic acids such as hydrochloric acid, hydrobromic acid, hydroiodic acid, sulfuric acid, nitric acid and phosphoric acid, or organic acids such as formic acid, acetic acid, propionic acid, oxalic acid, malonic acid, succinic acid, fumaric acid, maleic acid, lactic acid, malic acid, citric acid, tartaric acid, carbonic acid, picric acid, methanesulfonic acid, paratoluenesulfonic acid and glutamic acid; salts with inorganic bases such as sodium, potassium, magnesium, calcium and aluminum, organic bases such as methylamine, ethylamine, meglumine and ethanolamine, or basic amino acids such as lysine, arginine and ornithine; and ammonium salts.

The azabicyclo compound represented by the general formula (I) according to the present invention or a salt thereof can be synthesized in accordance with, for example, the method described in International Publication No. WO2011/004610.

The immune checkpoint molecule regulator in the present invention directly acts on immune checkpoint molecules, so that an in vivo antitumor immune response is induced in a cancer patient to control immune escape of a tumor.

Examples of the immune checkpoint molecule regulator include substances which promote the functions of costimulatory molecules (stimulatory costimulatory molecules), or substances which suppress the functions of coinhibitory molecules (inhibitory costimulatory molecules). Examples of the immune checkpoint molecules include B7 family (B7-1, B7-2, PD-L1, PD-L2, etc.), CD28 family (CTLA-4, PD-1, etc.), TNF superfamily (4-1BBL and OX40L), TNF receptor superfamily (4-1BB and OX40) molecules, and for the immune checkpoint molecule regulator, a substance targeted at immune checkpoint molecules can be used. Examples of the substance include PD-1 pathway antagonists, ICOS pathway agonists, CTLA-4 pathway antagonists, CD28 pathway agonists, BTLA pathway antagonists and 4-1BB pathway agonists.

In the present invention, the immune checkpoint molecule regulator is preferably at least one or more selected from the group consisting of a PD-1 pathway antagonist, an ICOS pathway agonist, a CTLA-4 pathway antagonist and a CD28 pathway agonist, more preferably at least one or more selected from the group consisting of a PD-1 pathway antagonist and a CTLA-4 pathway antagonist, still more preferably a PD-1 pathway antagonist from the viewpoint of suppressing side-effects.

The PD-1 pathway antagonist inhibits immunosuppressive signals from PD-1 expressed on T-cells, and PD-L1 or PD-L2 which is a ligand of PD-1, and examples of the PD-1 pathway antagonist include anti-PD-1 antibodies, anti-PD-L1 antibodies, anti-PD-L2 antibodies, PD-1 extracellular domains, PD-L1 extracellular domains, PD-L2 extracellular domains, PD-1-Ig (fusion proteins of PD-1 extracellular domains and FC regions of immunoglobulin (Ig)), PD-L1-Ig, PD-L2-Ig, PD-1, siRNA, PD-L1, siRNA and PD-L2 siRNA. The PD-1 pathway antagonist is preferably an anti-PD-1 antibody, an anti-PD-L1 antibody or an anti-PD-L2 antibody, more preferably an anti-PD-1 antibody or an anti-PD-L1 antibody, particularly preferably an anti-PD-1 antibody.

The CTLA-4 pathway antagonist inhibits immunosuppressive signals from CTLA-4 expressed on T-cells, and B7-1 (CD80) or B7-2 (CD86) which is a ligand of CTLA-4. The CTLA-4 pathway antagonist is preferably an anti-CTLA-4 antibody, a CTLA-4 extracellular domain, CTLA-4-Ig, an anti-B7-1 (CD80) antibody or an anti-B7-2 (CD86) antibody, more preferably an anti-CTLA-4 antibody or CTLA-4-Ig, particularly preferably an anti-CTLA-4 antibody.

In one of the aspects of the present invention, the immune checkpoint molecule regulator is preferably at least one or more selected from the group consisting of an anti-PD-1 antibody, an anti-PD-L1 antibody and an anti-CTLA-4 antibody.

Examples of the antibodies include immunoglobulins (IgA, IgD, IgE, IgG, IgM, IgY, etc.), Fab fragments, F(ab′)₂ fragments, single-stranded antibody fragments (scFv), single domain antibodies, and Diabody (Nat. Rev. Immunol., 6:343-357, 2006), which include monoclonal antibodies or polyclonal antibodies such as human antibodies, humanized antibodies, chimeric antibodies, mouse antibodies, llama antibodies and chicken antibodies.

Humanized IgG monoclonal antibodies or human IgG monoclonal antibodies are preferable.

The anti-PD-1 antibody in the present invention is nivolumab, pembrolizumab or the like, preferably nivolumab or pembrolizumab.

The anti-PD-L1 antibody in the present invention is atezolizumab, durvalumab, avelumab or the like, preferably atezolizumab, durvalumab or avelumab, more preferably atezolizumab.

The anti-CTLA-4 antibody in the present invention is ipilimumab, tremelimumab or the like, preferably ipilimumab.

The CTLA-4-Ig in the present invention is abatacept or the like, preferably abatacept.

These antibodies can be normally produced by a known method for preparation of an antibody.

The anti-PD-1 antibody is or will be sold as nivolumab or pembrolizumab, the PD-L1 antibody is or will be sold as atezolizumab, durvalumab or avelumab, the CTLA-4 antibody is or will be sold as ipilimumab or tremelimumab, and the CTLA-4-1g is or will be sold as abatacept. It is also possible use to these products.

In the present invention, when two or more immune checkpoint molecule regulators are used, for example, the anti-PD-1 antibody and the anti-CTLA-4 antibody can be used in combination, or a bispecific antibody capable of being bound to both PD-1 and CTLA-4 can be used. Examples of the bispecific antibody XmAb20717 (PD-1 x CTLA-4).

In the present invention, the dose per dosing day of the azabicyclo compound represented by the general formula (I) or a salt thereof is preferably 50 to 200%, more preferably 87.5 to 112.5%, particularly preferably 100%, of the recommended dosage of the singly administered azabicyclo compound represented by the general formula (I) or salt thereof, from the viewpoint of the enhancing effect on the antitumor effect of the immune checkpoint molecule regulator by the azabicyclo compound represented by the general formula (I). The recommended dosage in a human is preferably 80 to 320 mg/body/day, more preferably 140 to 180 mg/body/day, particularly preferably 160 mg/body/day.

In the present invention, the dose per dosing day of the immune checkpoint molecule regulator is preferably 50 to 100%, more preferably 100% of the recommended dosage of the singly administered immune checkpoint molecule regulator, from the viewpoint of the enhancing action on the antitumor effect of the immune checkpoint molecule regulator by the azabicyclo compound represented by the general formula (I).

Specifically, the recommended dosage of singly administered nivolumab is 2 mg/kg (body weight) per administration or 3 mg/kg (body weight) per administration, a dose approved in Japan, and thus the dose per dosing day of nivolumab in the present invention is preferably 1 to 3 mg/kg (body weight) per administration, more preferably 2 mg/kg (body weight) per administration or 3 mg/kg (body weight) per administration.

The recommended dosage of singly administered pembrolizumab is 2 mg/kg (body weight) per administration or 200 mg per administration, a dose approved in Japan, and thus the dose per dosing day of pembrolizumab in the present invention is preferably 1 to 2 mg/kg (body weight) per administration or 100 to 200 mg per administration, more preferably 2 mg/kg (body weight) or 200 mg per administration.

The recommended dosage of singly administered atezolizumab is 1200 mg per administration, a dose approved in US, and thus the dose per dosing day of atezolizumab in the present invention is preferably 600 to 1200 mg per administration, more preferably 1200 mg per administration.

The recommended dosage of singly administered avelumab or durvalumab is 10 mg/kg (body weight) per administration, a dose approved in US, and thus the dose per dosing day of avelumab or durvalumab in the present invention is preferably 5 to 10 mg/kg (body weight) per administration, more preferably 10 mg/kg (body weight) per administration.

The recommended dosage of singly administered ipilimumab is 3 mg/kg (body weight) per administration, a dose approved in Japan, and thus the dose per dosing day of ipilimumab in the present invention is preferably 1.5 to 3 mg/kg (body weight) per administration, more preferably 3 mg/kg (body weight) per administration.

In the present invention, the “recommended dosage” is a dose which is determined through a clinical test etc. and at which a maximum therapeutic effect is produced while safe use can be ensured without development of severe side-effects. Specifically, the “recommended dosage” is a dosage approved, recommended or suggested by public organizations or corporations such as Pharmaceuticals and Medical Devices Agency (PMDA), Food and Drug Administration (FDA) and European Medicines Agency (EMA), and described in attached documents, interview forms, treatment guidelines or the like, preferably a dose approved by a public organization selected from the group consisting of PMDA, FDA and EMA.

The administration schedule of the antitumor agent of the present invention can be appropriately selected according to the type of cancer, the stage of disease, etc.

The administration schedule of the azabicyclo compound represented by the general formula (I) or a salt thereof is preferably an administration schedule in which continuous administration for 5 days and cessation of administration for 2 days is repeated, specifically an administration schedule in which administration for 3 weeks using an administration method including administration for 5 days, followed by cessation of administration for 2 days in one week is set to one cycle, and the cycle is repeated.

The administration schedule of nivolumab is preferably an administration schedule in which administration is performed at intervals of 2 or 3 weeks.

The administration schedule of pembrolizumab, atezolizumab or ipilimumab is preferably an administration schedule in which administration is performed at intervals of 3 weeks.

The number of doses per day of the antitumor agent of the present invention is appropriately selected according to the type of cancer, the stage of disease, etc.

The number of doses per day of the azabicyclo compound represented by the general formula (I) or a salt thereof is preferably 1 or 2, more preferably 1. The number of doses per day of nivolumab, pembrolizumab, atezolizumab or ipilimumab is preferably 1.

The order of administration of the azabicyclo compound represented by the general formula (I) or a salt thereof and the immune checkpoint molecule regulator in the present invention can be appropriately selected according to the type of cancer, the stage of disease, etc., and these agents may be administered in any order, or in parallel. Here, when both the agents are not administered in parallel, the administration interval between both the agents can be appropriately selected as long as an enhancing effect on the antitumor effect is exhibited, and the administration interval is preferably 1 to 14 days, more preferably 1 to 7 days, still more preferably 1 to 5 days, particularly preferably 1 to 3 days.

The tumor to be targeted in the present invention is not particularly limited as long as an enhancing effect on the antitumor effect is exhibited, and the tumor is preferably a tumor against which the azabicyclo compound represented by the general formula (I) or a salt thereof exhibits an antitumor effect, more preferably a malignant tumor in which Hsp90 is involved.

Specific examples of the cancer to be targeted by the present invention include head and neck cancer, digestive organ cancer (esophagus cancer, gastric cancer, duodenal cancer, liver cancer, biliary tract cancer (gallbladder cancer, cholangiocarcinoma, etc.), pancreatic cancer, small intestine cancer, bowel cancer (colorectal cancer, colon cancer, rectal cancer, etc.), gastrointestinal stromal tumor, etc.), lung cancer (non-small cell lung cancer and small cell lung cancer), breast cancer, ovarian cancer, uterine cancer (cervical cancer, endometrial cancer, etc.), kidney cancer, bladder cancer, prostatic cancer and skin cancer. Here, the cancers include not only primary lesions but also cancers metastasizing to other organs (such as liver). In particular, from the viewpoint of antitumor effect and side-effects, the cancer is preferably head and neck cancer, digestive organ cancer, lung cancer, breast cancer, kidney cancer or skin cancer, more preferably digestive organ cancer, lung cancer or breast cancer, still more preferably bowel cancer, gastric cancer, gastrointestinal stromal tumor, lung cancer or breast cancer, with bowel cancer being particularly preferable. The antitumor agent of the present invention may be used for postoperative adjuvant chemotherapy to be carried out for prevention of recurrence after surgical removal of tumor, or may be used for preoperative adjuvant chemotherapy to be carried out before surgical removal of tumor.

As shown in Examples below, the azabicyclo compound represented by the general formula (I) or a salt thereof has immunostimulatory action. As used herein, the “immunostimulatory action” means the action of activating immune cells to induce cell division and production of various cytokines. The azabicyclo compound represented by the general formula (I) or a salt thereof has the action of activating T-cells, in particular, among immune cells. Here, the action of activating T-cells means, for example, increase in ratio of CD8 positive cells in tumor infiltrating leucocytes and/or decrease in ratio of regulatory T-cells in T-cells in tumor infiltrating leucocytes.

Therefore, the present invention relates to an immunostimulant containing an azabicyclo compound represented by the general formula (I) or a salt thereof as an active ingredient; an azabicyclo compound represented by the general formula (I) or a salt thereof for immunostimulation; and a method for immunostimulation, including the step of administering an effective amount of an azabicyclo compound represented by the general formula (I) or a salt thereof to a patient in need of immunostimulation. Examples of the immunostimulation target include humans and other mammals, e.g. monkeys, mice, rats, rabbits, dogs, cats, bovines, horses, pigs and sheep.

The immunostimulatory action of the azabicyclo compound represented by the general formula (1) or a salt thereof enables prevention and/or treatment of diseases which can be ameliorated by immunostimulation, such as various infectious diseases, immunodeficiency diseases, diseases caused by age-related decline in immune function and virus-related tumors.

Examples of the infectious diseases include parasitic infections (e.g. Trypanosoma protozoa, malaria protozoa and toxoplasma); bacterial infections (e.g. pneumococcus, tuberculosis bacteria, Staphylococcus aureus, anthrax bacteria, cholera bacteria, mycoplasma and pylori bacteria (Helicobacter pylori); and viral infections (e.g. human T-cell leukemia virus (HTLV-1), human immunodeficiency virus (HIV), papillomavirus (HPV), Epstein-Barr virus (EBV), cytomegalovirus (CMV), influenza virus (FLU), hepatitis B virus (HBV), herpesvirus, hepatitis C virus (HCV), SARS coronavirus, MERS coronavirus, dengue virus, Ebola virus).

Another aspect of the present invention relates to a pharmaceutical composition for preventing and/or treating an infectious disease by immunostimulation, including an azabicyclo compound represented by the general formula (I) or a salt thereof; an azabicyclo compound represented by the general formula (I) or a salt thereof for preventing and/or treating an infectious disease by immunostimulation; and a method for preventing and/or treating an infectious disease in a patient in need of the prevention and/or treatment, by immunostimulation, including administering to the patient an effective amount of an azabicyclo compound represented by the general formula (I) or a salt thereof.

Examples of the immunodeficiency diseases include congenital immunodeficiency diseases and acquired immunodeficiency diseases, particularly acquired immunodeficiency diseases caused by infection with human immunodeficiency virus (HIV).

Another aspect of the present invention relates to a pharmaceutical composition for preventing and/or treating an immunodeficiency disease by immunostimulation, including an azabicyclo compound represented by the general formula (I) or a salt thereof; an azabicyclo compound represented by the general formula (I) or a salt thereof for preventing and/or treating an immunodeficiency disease by immunostimulation; and a method for preventing and/or treating an immunodeficiency disease in a patient in need of the prevention and/or treatment, by immunostimulation, including administering to the patient an effective amount of an azabicyclo compound represented by the general formula (I) or a salt thereof.

Specific examples of the diseases caused by age-related decline in immune function include pneumonia.

Another aspect of the present invention relates to a pharmaceutical composition for preventing and/or treating a disease caused by age-related decline in immune function, by immunostimulation, including an azabicyclo compound represented by the general formula (I) or a salt thereof; an azabicyclo compound represented by the general formula (I) or a salt thereof for preventing and/or treating a disease caused by age-related decline in immune function, by immunostimulation; and a method for preventing and/or treating a disease caused by age-related decline in immune function in a patient in need of the prevention and/or treatment, by immunostimulation, including administering to the patient an effective amount of an azabicyclo compound represented by the general formula (I) or a salt thereof.

Specific examples of the virus-related tumors, i.e. tumors developed by viral infection, include Burkitt's lymphoma, hepatocellular cancer, cervical cancer, adult T-cell leukemia, Kaposi sarcoma and head and neck cancer.

Another aspect of the present invention relates to a pharmaceutical composition for preventing and/or treating a virus-related tumor by immunostimulation, including an azabicyclo compound represented by the general formula (I) or a salt thereof; an azabicyclo compound represented by the general formula (I) or a salt thereof for preventing and/or treating a virus-related tumor by immunostimulation; and a method for preventing and/or treating a virus-related tumor in a patient in need of the prevention and/or treatment, by immunostimulation, including administering to the patient an effective amount of an azabicyclo compound represented by the general formula (I) or a salt thereof.

In the present invention, the dose per dosing day of the azabicyclo compound represented by the general formula (I) or a salt thereof for immunostimulation is preferably 50 to 200%, more preferably 87.5 to 112.5%, particularly preferably 100%, of the recommended dosage of the singly administered azabicyclo compound represented by the general formula (I) or salt thereof, from the viewpoint of immunostimulatory action. The recommended dosage in a human is preferably 80 to 320 mg/body/day, more preferably 140 to 180 mg/body/day, particularly preferably 160 mg/body/day.

In the present invention, active ingredients in the azabicyclo compound represented by the general formula (I) or a salt thereof and the immune checkpoint molecule regulator may be separated and formulated in a plurality of dosage forms according to the formulations or administration schedules of the active ingredients, or integrated and formulated in one dosage form (i.e. formulated as a combination preparation). The preparations may be produced and sold in one package suitable for combination administration, or produced and sold in separate packages.

The formulations of the antitumor agent and the immunostimulant according to the present invention are not particularly limited, and can be appropriately selected according to the treatment purpose, and specific examples thereof include oral preparations (tablets, coated tablets, powders, granules, capsules, solutions, etc.), injections, suppositories, patches and ointments.

In the case of the azabicyclo compound represented by the general formula (I) or a salt thereof, oral preparations are preferable.

In the case of the anti-PD-1 antibody, anti-PD-L1 antibody or anti-CTLA-4 antibody, the aforementioned formulations are used, with injections being preferable.

For both the azabicyclo compound represented by the general formula (1) or a salt thereof and the immune checkpoint molecule regulator, the antitumor agent and the immunostimulant according to the present invention can be normally prepared by a known method using pharmaceutical acceptable carriers depending on the formulations of the agents. Examples of the carriers include various carriers which are commonly used for normal pharmaceutical agents, e.g. excipients, binders, disintegrants, lubricants, diluents, solubilizers, suspending agents, tonicity agents, pH adjustors, buffers, stabilizers, coloring agents, flavor improving agents and odor improving agents.

The present invention also relates to an antitumor effect enhancing agent for enhancing the antitumor effect of an immune checkpoint molecule regulator on a cancer patient, the antitumor effect enhancing agent containing an azabicyclo compound represented by the general formula (I) or a salt thereof as an active ingredient. The antitumor effect enhancing agent has a preparation form identical to that of the aforementioned antitumor agent.

The present invention also relates to an antitumor effect enhancing agent for enhancing the antitumor effect of an azabicyclo compound represented by the general formula (I) or a salt thereof on a cancer patient, the antitumor effect enhancing agent containing an immune checkpoint molecule regulator as an active ingredient. The antitumor effect enhancing agent has a preparation form identical to that of the aforementioned antitumor agent.

The present invention also relates to an antitumor agent for treating a cancer patient given an immune checkpoint molecule regulator, the antitumor agent containing an azabicyclo compound represented by the general formula (I) or a salt thereof. The antitumor agent has the aforementioned preparation form.

The present invention also relates to an antitumor agent for treating a cancer patient given an azabicyclo compound represented by the general formula (I) or a salt thereof, the antitumor agent containing an immune checkpoint molecule regulator. The antitumor agent has the aforementioned preparation form.

The “treatment” includes postoperative adjuvant chemotherapy to be carried out for prevention of recurrence after surgical removal of tumor, and preoperative adjuvant chemotherapy to be carried out before surgical removal of tumor.

The present invention also relates to an antitumor agent containing an azabicyclo compound represented by the general formula (I) or a salt thereof, used in combination with an immune checkpoint molecule regulator for a cancer patient. The antitumor agent has the aforementioned preparation form.

The present invention also relates to an antitumor agent containing an immune checkpoint molecule regulator, used in combination with an antitumor agent containing an azabicyclo compound represented by the general formula (I) or a salt thereof for a cancer patient. The antitumor agent has the aforementioned preparation form.

The present invention also relates to a kit preparation including an antitumor agent containing an azabicyclo compound represented by the general formula (I) or a salt thereof; and a written instruction which indicates that an azabicyclo compound represented by the general formula (I) or a salt thereof and an immune checkpoint molecule regulator are administered in combination to a cancer patient.

Here, the “written instruction” is not limited as long as the aforementioned doses are specified. The written instruction may be legally binding or non-binding, and is preferably one in which the aforementioned doses are recommended. Specific examples of the written instruction include attached documents and brochures. The kit preparation including a written instruction may be one in which a written instruction is printed or attached on a kit preparation package, or one in which a written instruction is enclosed together with an antitumor agent in a kit preparation package.

### EXAMPLES

The present invention will now be described in further detail by way of examples, which should not be construed as limiting the present invention, and those skilled in the art can make many modifications within the technical concept of the present invention.

### Example 1: Effect of Combination of Compound 1 and Anti-Mouse PD-1 Antibody in Bowel Cancer Cell Transplanted Model

Mouse bowel cancer cell line MC38 was obtained from Dr. Yoshihiro Hayakawa (University of Toyama, Toyama, Japan), and cultured in a RPMI1640 (Sigma-Aldrich) medium (culture solution) containing 10% fetal bovine serum (FBS). MC38 was subcultured at a ratio of 1:3 to 1:5 once or twice a week in an incubator with 5% CO₂ at 37° C.

A cell suspension with a concentration of 2×10⁶ cells/0.1 mL was transplanted subcutaneously in the vicinity of the right backmost rib bone of a 6-week-old C57BL/6JJcI mouse (CLEA Japan, Inc.).

The tumor was grown until reaching a tumor volume (TV) of 50 to 300 mm³ after the transplantation. Degimatic Caliper was used for tumor diameter measurement. The major diameter and the minor diameter of the tumor were measured, and TV was calculated from the following equation.

$$TV ( {mm}^{3} ) = major diameter ( mm ) \times minor diameter ( mm ) \times minor diameter ( mm ) / 2$$

By a stratified random allocation method using TV as an index, 6 animals were allocated to each group. The day when grouping (n=6) was performed was defined as Day 0.

The relative tumor volume (RTV) and the relative tumor volume change ratio (T/C) were calculated from TV.

RTV and T/C were calculated from the following equation.

$$RTV = TV on measurement day / TV on grouping day T / C ( \% ) = ( average R TV of dosed group ) / average R TV of control group ) \times 100$$

An electronic balance for animals was used for body weight measurement. The body weight change ratio on the nth day (BWCn) was calculated from the body weight on the nth day (BWn) in accordance with the following equation.

$$Body weight change ratio BWCn ( \% ) = ( BWn - BW 0 ) / BW 0 \times 100$$

An appropriate amount of water for injection described in the Japanese Pharmacopeia was added for adjusting hypromellose concentration to 0.5 w/v %, and the mixture was then stirred with a stirrer to completely dissolve hypromellose, to prepare a 0.5% aqueous hypromellose solution. Compound 1 was ground with an agate mortar, and then suspended to a predetermined concentration with the 0.5% aqueous hypromellose solution, and ultrasonic treatment was performed to obtain a homogeneous suspension. The suspension containing Compound 1 was orally administered once a day over 5 consecutive days at a dose of 10 mg/kg/day or 14 mg/kg/day, followed by cessation of administration for 2 days, and these processes were repeated three times.

The anti-mouse PD-1 antibody (anti-mPD-1 Ab) was prepared by diluting InVivoMab anti-mouse PD-1 (CD279) (Clone: RMP-1-14, Bio X cell Co.) to a predetermined concentration with Otsuka Normal Saline immediately before administration. On the first day of administration (day 1), the diluted product was intraperitoneally administered as the anti-mouse PD-1 antibody at a dose of 0.05 mg/body.

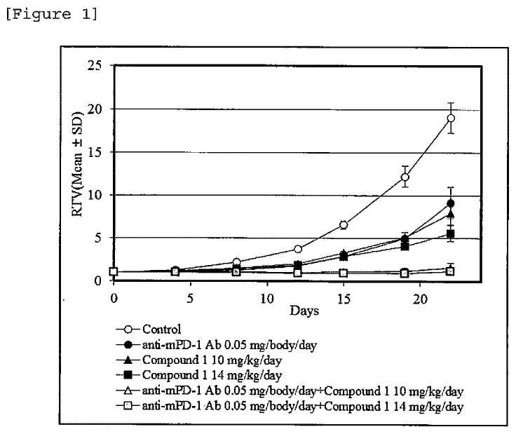

The results are shown in FIG. 1 and Table 1.

TABLE 1

| | | | | | RTV | | BWC(%) |
|---|---|---|---|---|---|---|---|
| | Dose | | Schedule | | Day 22 | T/C | Day 22 |
| Group | (/day) | | (day) | Route | (mean ± SD) | (%) | Mean |
| Control | — | | 1-5, 8-12, 15-19 | p.o. | 19.02 ± 1.77 | 100.0 | 11.8 |
| Anti-mPD-1 Ab | 0.05 | mg/body | 1 | i.p. | 9.18 ± 1.85 ** | 48.3 | 15.5 |
| Compound 1 | 10 | mg/kg | 1-5, 8-12, 15-19 | p.o. | 7.93 ± 1.29 ** | 41.7 | 13.5 |
| Compound 1 | 14 | mg/kg | 1-5, 8-12, 15-19 | p.o. | 5.59 ± 0.92 ** | 29.4 | 12.8 |

| | | | | | | |
|---|---|---|---|---|---|---|
| Anti-mPD-1 Ab + | 0.05 mg/body + | 1 + | i.p + | 1.58 ± 0.55 **^{##$$} | 8.3 | 12.7 |
| Compound 1 | 10 mg/kg | 1-5, 8-12, 15-19 | p.o. | | | |
| Anti-mPD-1 Ab + | 0.05 mg/body + | 1 + | i.p. + | 1.16 ± 0.41 **^{##$$} | 6.1 | 15.1 |
| Compound 1 | 14 mg/kg | 1-5, 8-12, 15-19 | p.o. | | | |

** p < 0.01 with Aspin-Welch's t-test as compared with the Control group.
^{##} p < 0.01 with Aspin-Welch's t-test as compared with the anti-mouse PD-1 antibody (anti-mPD-1 Ab) group.
^{$$} p < 0.01 with Aspin-Welch's t-test as compared with the Compound 1 group.
SD: standard deviation

RTV of each group on Day 22 was analyzed by the Aspin-Welch's t-test, and the result showed that both the single-agent group to which Compound 1 group or the anti-mouse PD-1 antibody was administered, and the combination administration group to which Compound 1+anti-mouse PD-1 antibody were administered exhibited an antitumor effect with significantly lower RTV as compared to the control group. Further, the combination administration group to which Compound 1+anti-mouse PD-1 antibody were administered had significantly lower RTV and exhibited a higher antitumor effect as compared to the single-agent group to which Compound 1 or the anti-mouse PD-1 antibody was administered.

The average body weight change ratio of the combination administration group indicated no enhanced toxicity as compared to the single-agent group to which Compound 1 or the anti-mouse PD-1 antibody alone was administered.

### Example 2: Immunostimulatory Action of Compound 1 in Bowel Cancer Cell Transplanted Model

Mouse bowel cancer cell line MC38 was cultured using a RPMI1640 (Waco Pure Chemical Industries, Ltd.) medium containing 10% FBS. MC38 was subcultured at a ratio of 1:5 to 1:10 once or twice a week in an incubator with 5% CO₂ at 37° C.

A cell suspension with a concentration of 2×10⁶ cells/0.1 mL was transplanted subcutaneously in the vicinity of the right backmost rib bone of a 6-week-old C57BL/6NJCrI mouse (Charles River Laboratories Japan Inc.).

The tumor was grown until reaching a tumor volume (TV) of 110 to 360 mm³ after the transplantation. Degimatic Caliper was used for tumor diameter measurement. The major diameter and the minor diameter of the tumor were measured, and TV was calculated from the following equation.

$$TV ( {mm}^{3} ) = major diameter ( mm ) \times minor diameter ( mm ) \times minor diameter ( mm ) / 2$$

By a stratified random allocation method using TV as an index, 5 animals were allocated to each group. The day when grouping (n=5) was performed was defined as Day 1.

Agents prepared by the same method as described in Example 1 were used for administration. Anti-mouse PD-1 antibody (anti-mPD-1 Ab), CD279(PD-1)Monoclonal Antibody (RMP1-14), eBioscience (Thermo Fisher Scientific), was used as an anti-mouse PD-1 antibody, and intraperitoneally administered at a dose of 0.05 mg/body on the first day of administration (Day 1). Compound 1 was orally administered at a dose of 14 mg/kg/day once a day on Days 1-5 and 8-9.

For detecting expression of immunity-related factors, flow cytometric analysis was performed for immunological monitoring. On Day 10, the tumor was sampled, and tumor infiltrating lymphocytes were then prepared using Tumor Dissociation Kit, mouse (Miltenyi Biotec). Thereafter, the antibody was stained using FoxP3/Transcription Factor Staining Buffer Set (thermo Fisher Scientific), and analysis was then performed using Flow Cytometer FACSVerse (BD Bioscience). For the staining, CD45 Monoclonal Antibody (30-F11), eFluor 450, eBioscience™ (Thermo Fisher Scientific), FoxP3 Monoclonal Antibody (FJK-16s), PE, eBioscience™ (Thermo Fisher Scientific), CD4 Monoclonal Antibody (GK1.5), PE-Cyanine7, eBioscience™ (Thermo Fisher Scientific) and CD25 Monoclonal Antibody (PC61.5), APC, eBioscience™ (Thermo Fisher Scientific), Brilliant Violet 510™ anti-mouse CD90.2 Antibody (BioLegend) and CD8a MonoClonal Antibody (53-6.7), FITC, eBioscience™ (BioLegend) were used.

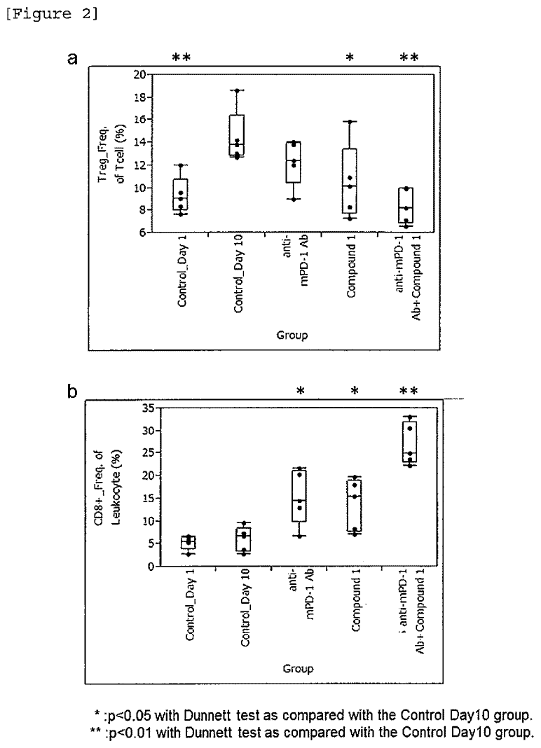

The results are shown in FIG. 2.

The ratio of CD8 positive cells (CD45 positive, CD90.2 positive and CD8 positive cells) in leucocytes (CD45 positive cells) of each group on Day 10 was analyzed by the Dunnett test, and the result showed that the Compound 1-administered group had a significantly higher ratio of CD8 positive cells in leucocytes as compared to the control group on Day 10. The ratio of regulatory T-cells (CD45 positive, CD90.2 positive, CD4 positive, CD25 positive and FoxP3 positive cells) in T-cells (CD45 positive and CD90.2 positive cells) of each group on Day 10 was analyzed by the Dunnett test, and the result showed that the Compound 1-administered group had a significantly lower ratio of regulatory T-cells in T-cells as compared to the control group on Day 10.

The above results showed that Compound 1 shows immunostimulatory activity.

### Example 3: Effect of Combination of Compound 1 and Anti-Mouse PD-L1 Antibody or Anti-Mouse CTLA-4 Antibody in Bowel Cancer Cell Transplanted Model

The same procedure as described in Example 1 was carried out except that the anti-mouse PD-L1 antibody or the anti-CTLA-4 antibody was used instead of the anti-mouse PD-1 antibody.

The anti-mouse PD-L1 antibody (anti-mPD-L1 Ab) was prepared by diluting InVivoMab anti-mouse PD-L1 (B7-H1) (Clone: 10F.9G2, Bio X cell Co.) to a predetermined concentration with Otsuka Normal Saline immediately before administration. On the first day of administration (day 1), the diluted product was intraperitoneally administered using the anti-mouse PD-L1 antibody at a dose of 0.05 mg/body. The anti-mouse CTLA-4 antibody (anti-mCTLA-4 Ab) was prepared by diluting InVivoMab anti-mouse CTLA-4 (CD152) (Clone: 9H10, Bio X cell Co.) to a predetermined concentration with Otsuka Normal Saline immediately before administration. On the first day of administration (day 1), the diluted product was intraperitoneally administered using the anti-mouse CTLA-4 antibody at a dose of 0.05 mg/body.

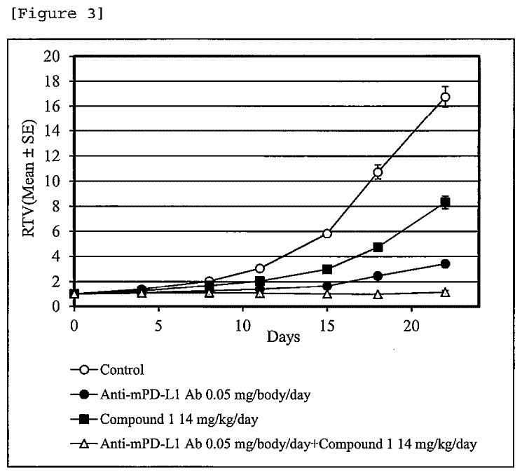

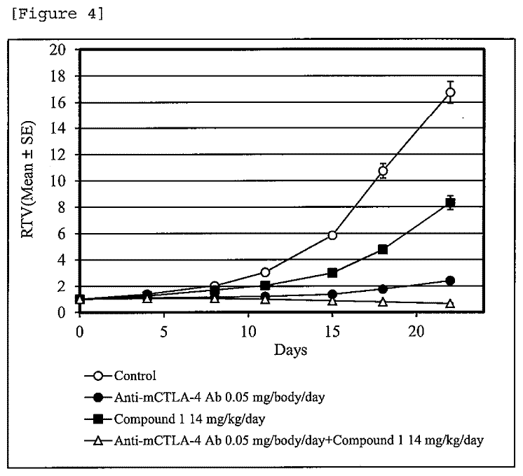

The results are shown in FIGS. 3 and 4 and Table 2.

TABLE 2

| | | | | | RTV | | BWC(%) |
|---|---|---|---|---|---|---|---|
| | Dose | | Schedule | | Day 22 | T/C | Day 22 |
| Group | (/day) | | (day) | Route | (mean ± SE) | (%) | Mean |
| Control | — | | 1-5, 8-12, 15-19 | p.o. | 16.75 ± 0.81 | 100.0 | 5.88 |
| Anti-mPD-L1 Ab | 0.05 | mg/body | 1 | i.p. | 3.43 ± 0.25 ** | 20.5 | 7.95 |
| Anti-mCTLA-4 Ab | 0.05 | mg/body | 1 | i.p. | 2.41 ± 0.21 ** | 14.4 | 7.17 |
| Compound 1 | 14 | mg/kg | 1-5, 8-12, 15-19 | p.o. | 8.32 ± 0.51 ** | 49.7 | 6.63 |

| | | | | | | |
|---|---|---|---|---|---|---|
| Anti-mPD-L1 Ab + | 0.05 mg/body + | 1 + | i.p + | 1.14 ± 0.13 **^{##$$} | 6.8 | 7.01 |
| Compound 1 | 14 mg/kg | 1-5, 8-12, 15-19 | p.o. | | | |
| Anti-mCTLA-4 Ab + | 0.05 mg/body + | 1 + | i.p. + | 0.65 ± 0.03 **^{&&$$} | 3.9 | 5.97 |
| Compound 1 | 14 mg/kg | 1-5, 8-12, 15-19 | p.o. | | | |

** p < 0.01 with Aspin-Welch's t-test as compared with the Control group.
^{##} p < 0.01 with Aspin-Welch's t-test as compared with the anti-mouse PD-L1 antibody (anti-mPD-L1 Ab) group.
^{&&} p < 0.01 with Aspin-Welch's t-test as compared with the anti-mouse CTLA-4 antibody (anti-mCTLA-4 Ab) group.
^{$$} p < 0.01 with Aspin-Welch's t-test as compared with the Compound 1 group.
SE: standard error

RTV of each group on Day 22 was analyzed by the Aspin-Welch's t-test, and the result showed that the single-agent group to which Compound 1, the anti-mouse PD-L1 antibody or the anti-mouse CTLA-4 antibody was administered, and the combination administration group to which Compound 1+anti-mouse PD-L1 antibody or the anti-mouse CTLA-4 antibody were administered exhibited an antitumor effect with significantly lower RTV as compared to the control group. Further, the combination administration group to which Compound 1+anti-mouse PD-L1 antibody were administered had significantly lower RTV and exhibited a higher antitumor effect as compared to the single-agent group to which Compound 1 or the anti-mouse PD-L1 antibody was administered. The combination administration group to which Compound 1+anti-mouse CTLA-4 antibody were administered had significantly lower RTV and exhibited a higher antitumor effect as compared to the single-agent group to which Compound 1 or the anti-mouse CTLA-4 antibody was administered.

The average body weight change ratio of the combined administration group indicated no enhanced toxicity as compared to the single-agent group to which Compound 1, the anti-mouse PD-L1 antibody or the anti-mouse CTLA-4 antibody alone was administered.

1. An antitumor agent comprising an azabicyclo compound or a salt in combination with an immune checkpoint molecule regulator,
the azabicyclo compound being a compound represented by the general formula (I): 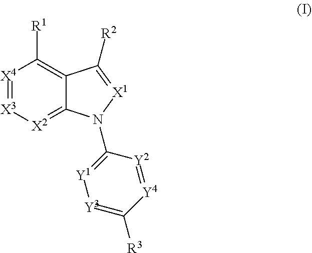
wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.

2. The antitumor agent according to claim 1, wherein the azabicyclo compound is a compound of the general formula (I) in which X¹ is CH or N;
X² is N, and X³ and X⁴ are CH;
Y¹ and Y³ are CH, one or two of Y² and Y⁴ are C—R⁴, and the other is CH;
R¹ is any one of an optionally substituted 1H-imidazol-1-yl group, an optionally substituted pyrazol-4-yl group, an optionally substituted thiophen-3-yl group, an optionally substituted furan-2-yl group, an optionally substituted pyridin-3-yl group, an optionally substituted pyridin-4-yl group, an optionally substituted indol-5-yl group, an optionally substituted 1H-pyrrolo[2,3-b]pyridin-5-yl group, an optionally substituted benzofuran-2-yl group, an optionally substituted quinolin-3-yl group and an optionally substituted 5,6,7,8-tetrahydroquinon-3-yl group;
R² is an alkyl group having 1 to 6 carbon atoms and optionally having a halogen atom, or an alkenyl group having 2 to 6 carbon atoms;
R³ is —CO—R⁵;
R⁴ is an alkyl group having 1 to 6 carbon atoms and optionally having a halogen atom, a mono- or di-(C1-C6 alkyl)amino group or a monocyclic 5- to 7-membered saturated heterocyclic group having 1 or 2 heteroatoms selected from the group consisting of N, S and O, an alkoxy group having 1 to 6 carbon atoms, —N(R⁶)(R⁷), —S—R³ or —CO—R⁹;
R⁵ is an amino group, or a mono- or di-(C1-C6 alkyl)amino group;
R⁶ is a hydrogen atom, or an optionally substituted alkyl group having 1 to 6 carbon atoms;
R⁷ is a hydrogen atom, an optionally substituted alkyl group having 1 to 6 carbon atoms, an optionally substituted cycloalkyl group having 3 to 7 carbon atoms, an optionally substituted aralkyl group having 7 to 12 carbon atoms, an optionally substituted aromatic hydrocarbon group having 6 to 14 carbon atoms, an optionally substituted monocyclic or dicyclic saturated heterocyclic group having 1 to 4 heteroatoms selected from the group consisting of N, S and O, or an optionally substituted monocyclic or dicyclic unsaturated heterocyclic group having 1 to 4 heteroatoms selected from the group consisting of N, S and O, or R⁶ and R⁷ form a 5- to 7-membered saturated heterocyclic group, together with the nitrogen atom to which they are attached;
R⁸ is an optionally substituted cycloalkyl group having 3 to 7 carbon atoms, or an optionally substituted aromatic hydrocarbon group having 6 to 14 carbon atoms; and
R⁹ is a hydrogen atom, a hydroxyl group, an amino group, or a mono- or a di-(C1-C6 alkyl)amino group.

3. The antitumor agent according to claim 1, wherein the azabicyclo compound is 3-ethyl-4-{3-isopropyl-4-(4-(1-methyl-1H-pyrazol-4-yl)-1H-imidazol-1-yl)-1H-pyrazolo[3,4-b]pyridin-1-yl}benzamide.

4. The antitumor agent according to claim 1, wherein the immune checkpoint molecule regulator is at least one selected from the group consisting of a PD-1 pathway antagonist, an ICOS pathway agonist, a CTLA-4 pathway antagonist and a CD28 pathway agonist.

5. The antitumor agent according to claim 1, wherein the immune checkpoint molecule regulator is at least one selected from the group consisting of a PD-1 pathway antagonist, a CTLA-4 pathway antagonist and a CD28 pathway agonist.

6. The antitumor agent according to claim 1, wherein the immune checkpoint molecule regulator is at least one selected from the group consisting of a PD-1 pathway antagonist and a CTLA-4 pathway antagonist.

7. The antitumor agent according to claim 1, wherein the immune checkpoint molecule regulator is a PD-1 pathway antagonist.

8. The antitumor agent according to claim 7, wherein the PD-1 pathway antagonist is at least one selected from the group consisting of an anti-PD-1 antibody, an anti-PD-L1 antibody and an anti-PD-L2 antibody.

9. The antitumor agent according to claim 8, wherein the anti PD-1-antibody is at least one selected from the group consisting of nivolumab and pembrolizumab, and the anti-PD-L1 antibody is at least one selected from the group consisting of atezolizumab, durvalumab and avelumab.

10. The antitumor agent according to claim 4, wherein the CTLA-4 pathway antagonist is an anti-CTLA-4 antibody.

11. The antitumor agent according to claim 10, wherein the anti-CTLA-4 antibody is at least one selected from the group consisting of ipilimumab and tremelimumab.

12. A method for treating a tumor, comprising administering to a subject in need thereof an azabicyclo compound or a salt thereof and an immune checkpoint molecule regulator in combination, the azabicyclo compound being represented by the general formula (I): 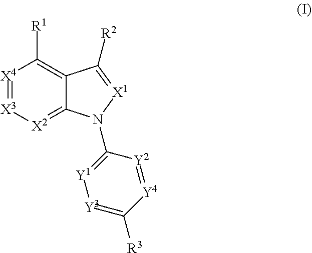
wherein X¹ represents CH or N;
any one of X², X³ and X⁴ represents N, and the others represent CH;
any one or two of Y¹, Y², Y³ and Y⁴ represent C—R⁴, and the others are the same or different and represent CH or N;
R¹ represents an optionally substituted monocyclic or bicyclic unsaturated heterocyclic group having 1 to 4 heteroatoms selected from N, S and O;
R² represents a hydrogen atom, an optionally substituted alkyl group having 1 to 6 carbon atoms or an optionally substituted alkenyl group having 2 to 6 carbon atoms;
R³ represents a cyano group or —CO—R⁵;
R⁴(s) are the same or different and represent a hydrogen atom, a halogen atom, a cyano group, an optionally substituted alkyl group having 1 to 6 carbon atoms, an alkenyl group having 2 to 6 carbon atoms, an alkoxy group having 1 to 6 carbon atoms, an aromatic hydrocarbon group, —N(R⁶)(R⁷), —S—R³ or —CO—R⁹;
R⁵ represents an amino group optionally having a hydroxyl group, or an optionally substituted mono- or di-alkylamino group;
R⁶ and R⁷ are the same or different and represent a hydrogen atom, an optionally substituted alkyl group having 1 to 6 carbon atoms, a halogenoalkyl group having 1 to 6 carbon atoms, an optionally substituted cycloalkyl group having 3 to 7 carbon atoms, an optionally substituted aralkyl group, an optionally substituted aromatic hydrocarbon group, an optionally substituted saturated heterocyclic group or an optionally substituted unsaturated heterocyclic group, or R⁶ and R⁷ optionally form a saturated heterocyclic group, together with the nitrogen atom to which they are attached;
R³ represents an optionally substituted cycloalkyl group having 3 to 7 carbon atoms, or an optionally substituted aromatic hydrocarbon group; and
R⁹ represents a hydrogen atom, a hydroxyl group, an amino group optionally having a hydroxyl group, or an optionally substituted mono- or di-alkylamino group.

13. The method according to claim 12, wherein the azabicyclo compound is a compound of the general formula (I) in which X¹ is CH or N;
X² is N, and X³ and X⁴ are CH;
Y¹ and Y³ are CH, one or two of Y² and Y⁴ are C—R⁴, and the other is CH;
R¹ is any one of an optionally substituted 1H-imidazol-1-yl group, an optionally substituted pyrazol-4-yl group, an optionally substituted thiophen-3-yl group, an optionally substituted furan-2-yl group, an optionally substituted pyridin-3-yl group, an optionally substituted pyridin-4-yl group, an optionally substituted indol-5-yl group, an optionally substituted 1H-pyrrolo[2,3-b]pyridin-5-yl group, an optionally substituted benzofuran-2-yl group, an optionally substituted quinolin-3-yl group and an optionally substituted 5,6,7,8-tetrahydroquinon-3-yl group;
R² is an alkyl group having 1 to 6 carbon atoms and optionally having a halogen atom, or an alkenyl group having 2 to 6 carbon atoms;
R³ is —CO—R⁵;
R⁴ is an alkyl group having 1 to 6 carbon atoms and optionally having a halogen atom, a mono- or di-(C1-C6 alkyl)amino group or a monocyclic 5- to 7-membered saturated heterocyclic group having 1 or 2 heteroatoms selected from the group consisting of N, S and O, an alkoxy group having 1 to 6 carbon atoms, —N(R⁶)(R⁷), —S—R³ or —CO—R⁹;
R⁵ is an amino group, or a mono- or di-(C1-C6 alkyl)amino group;
R⁶ is a hydrogen atom, or an optionally substituted alkyl group having 1 to 6 carbon atoms;
R⁷ is a hydrogen atom, an optionally substituted alkyl group having 1 to 6 carbon atoms, an optionally substituted cycloalkyl group having 3 to 7 carbon atoms, an optionally substituted aralkyl group having 7 to 12 carbon atoms, an optionally substituted aromatic hydrocarbon group having 6 to 14 carbon atoms, an optionally substituted monocyclic or dicyclic saturated heterocyclic group having 1 to 4 heteroatoms selected from the group consisting of N, S and O, or an optionally substituted monocyclic or dicyclic unsaturated heterocyclic group having 1 to 4 heteroatoms selected from the group consisting of N, S and O, or R⁶ and R⁷ form a 5- to 7-membered saturated heterocyclic group, together with the nitrogen atom to which they are attached;
R⁸ is an optionally substituted cycloalkyl group having 3 to 7 carbon atoms, or an optionally substituted aromatic hydrocarbon group having 6 to 14 carbon atoms; and
R⁹ is a hydrogen atom, a hydroxyl group, an amino group, or a mono- or a di-(C1-C6 alkyl)amino group.

14. The method according to claim 12, wherein the azabicyclo compound is 3-ethyl-4-{3-isopropyl-4-(4-(1-methyl-1H-pyrazol-4-yl)-1H-imidazol-1-yl)-1H-pyrazolo[3,4-b]pyridin-1-yl}benzamide.

15. The method according to claim 12, wherein the immune checkpoint molecule regulator is at least one selected from the group consisting of a PD-1 pathway antagonist, an ICOS pathway agonist, a CTLA-4 pathway antagonist and a CD28 pathway agonist.

16. The method according to claim 12, wherein the immune checkpoint molecule regulator is at least one selected from the group consisting of a PD-1 pathway antagonist, a CTLA-4 pathway antagonist and a CD28 pathway agonist.

17. The method according to claim 12, wherein the immune checkpoint molecule regulator is at least one selected from the group consisting of a PD-1 pathway antagonist and a CTLA-4 pathway antagonist.

18. The method according to claim 12, wherein the immune checkpoint molecule regulator is a PD-1 pathway antagonist.

19. The method according to claim 18, wherein the PD-1 pathway antagonist is at least one selected from the group consisting of an anti-PD-1 antibody, an anti-PD-L1 antibody and an anti-PD-L2 antibody.

20. The method according to claim 19, wherein the anti PD-1-antibody is at least one selected from the group consisting of nivolumab and pembrolizumab, and the anti-PD-L1 antibody is at least one selected from the group consisting of atezolizumab, durvalumab and avelumab.

21. The method according to claim 15, wherein the CTLA-4 pathway antagonist is an anti-CTLA-4 antibody.

22. The method according to claim 21, wherein the anti-CTLA-4 antibody is at least one selected from the group consisting of ipilimumab and tremelimumab.
